(19) United States
(12) Patent Application Publication
(10) Pub. No.: US 2002/0076700 A1
(43) Pub. Date: Jun. 20, 2002

(54) Novel polypeptides and nucleic acids encoding same

(76) Inventor: Richard A. Shimkets, West Haven, CT (US)

Correspondence Address: Ivor R. Elrifi, Ph.D., MINTZ, LEVIN, COHN, FERRIS,, GLOVSKY and POPEO, P.C., One Financial Center, Boston, MA, 02111, US

(21) Appl. No.: 09/740,668

(22) Filed: Dec. 18, 2000

Related U.S. Application Data
(63) Continuation-in-part of application No. PCT/US99/29845, filed on Dec. 17, 1999.
(63) Continuation-in-part of application No. 09/465,512, filed on Dec. 16, 1999.
(60) Provisional application No. 60/113,485, filed on Dec. 21, 1998.
(60) Provisional application No. 60/112,837, filed on Dec. 18, 1998.

(51) Int. Cl. C12Q 1/68; C07H 21/04; C12N 9/00; C12P 21/02; C12N 5/06; C07K 14/435

(52) U.S. Cl. 435/6; 435/69.1; 435/183; 435/320.1; 435/325; 530/350; 536/23.1

(57) ABSTRACT

Disclosed are novel secreted polypeptides, nucleic acids encoding same, as well as antibodies to the polypeptides.

### RELATED APPLICATIONS

This application claims priority to PCT/US99/29854, filed Dec. 17, 1999; U.S. Ser. No. 09/465,512, filed Dec. 16, 1999; USSN 60/113,485, filed Dec. 21, 1998; and U.S. Ser. No. 60/112,837, filed Dec. 18, 1998, each of which is incorporated herein by reference in its entirety.

### FIELD OF THE INVENTION

The invention generally relates to nucleic acids and polypeptides and in particular to nucleic acids encoding secreted or membrane-associated proteins.

### BACKGROUND OF THE INVENTION

Eukaryotic cells are subdivided by membranes into multiple functionally distinct compartments that are referred to as organelles. Each organelle includes proteins essential for its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Only a limited number of genes encoding human membrane-bound and secreted proteins have been identified. Examples of known secreted proteins include human insulin, interferon, interleukins, transforming growth factor-beta, human growth hormone, erythropoietin, and lymphokines.

### SUMMARY OF THE INVENTION

The present invention is based, in part, upon the discovery of 23 nucleic acids encoding novel secreted human proteins (collectively referred to herein as SECX nucleic acid sequences) and the polypeptides, termed SECX polypeptides or proteins, encoded by these nucleic acid sequences.

In one aspect, the invention includes an isolated SECX nucleic acid molecule which includes a nucleotide sequence at least 85% similar to the nucleotide sequence of SEQ ID NOs; 1, 3, 5, 9, 11, 13, 15, 17, 19, 21, 23, 25, 27, 29, 31, 33, 35, 37, 39, 41, 43, 45, 47, 149, 151, 153, 155, or 156, or a complement thereof.

The invention also includes an isolated polypeptide having an amino acid sequence at least 80% homologous to a SECX polypeptide which includes the amino acid sequence of SEQ ID NOs 2, 4, 6, 8, 10, 12, 14, 16, 20, 22, 24, 26, 28, 30, 32, 34, 36, 38, 40, 42, 44, 46, 48, 150, 152, or 154: or a fragment having at least 15 amino acids of these amino acid sequences. Also included is a naturally occurring polypeptide variant of a SECX polypeptide, wherein the polypeptide is encoded by a nucleic acid molecule which hybridizes under stringent conditions to a nucleic acid molecule consisting of a SECX nucleic acid molecule.

Also included in the invention is an antibody which selectively binds to a SECX polypeptide which includes the amino acid sequence of SEQ ID NOs 2, 4, 6, 8, 10, 12, 14, 16, 20, 22, 24, 26, 28, 30, 32, 34, 36, 38, 40, 42, 44, 46, 48, 150, 152, or 154.

The invention further includes a method for producing a SECX polypeptide by culturing a host cell expressing one of the herein described SECX nucleic acids under conditions in which the nucleic acid molecule is expressed.

The invention also includes methods for detecting the presence of a SECX polypeptide or nucleic acid in a sample from a mammal, e.g., a human, by contacting a sample from the mammal with an antibody which selectively binds to one of the herein described polypeptides, and detecting the formation of reaction complexes including the antibody and the polypeptide in the sample. Detecting the formation of complexes in the sample indicates the presence of the polypeptide in the sample.

The invention further includes a method for detecting or diagnosing the presence of a disease, e.g., a pathological condition, associated with altered levels of a polypeptide having an amino acid sequence at least 80% identical to a SECX polypeptide in a sample. The method includes measuring the level of the polypeptide in a biological sample from the mammalian subject, e.g., a human, and comparing the level detected to a level of the polypeptide present in normal subjects, or in the same subject at a different time, e.g., prior to onset of a condition. An increase or decrease in the level of the polypeptide as compared to normal levels indicates a disease condition.

Also included in the invention is a method of detecting the presence of a SECX nucleic acid molecule in a sample from a mammal, e.g, a human. The method includes contacting the sample with a nucleic acid probe or primer which selectively hybridizes to the nucleic acid molecule and determining whether the nucleic acid probe or primer binds to a nucleic acid molecule in the sample. Binding of the nucleic acid probe or primer indicates the nucleic acid molecule is present in the sample.

The invention further includes a method for detecting or diagnosing the presence of a disease associated with altered levels of a SECX nucleic acid in a sample from a mammal, e.g,. a human. The method includes measuring the level of the nucleic acid in a biological sample from the mammalian subject and comparing the level detected to a level of the nucleic acid present in normal subjects, or in the same subject at a different time. An increase or decrease in the level of the nucleic acid as compared to normal levels indicates a disease condition.

The invention also includes a method of treating a pathological state in a mammal, e.g,. a human, by administering to the subject a SECX polypeptide to the subject in an amount sufficient to alleviate the pathological condition. The polypeptide has an amino acid sequence at least 80% identical to a polypeptide which includes the amino acid sequence of SEQ ID NOs 2, 4, 6, 8, 10, 12, 14, 16, 20, 22, 24, 26, 28, 30, 32, 34, 36, 38, 40, 42, 44, 46, 48, 150, 152, or 154, or a biologically active fragment thereof.

Alternatively, the mammal may be treated by administering an antibody as herein described in an amount sufficient to alleviate the pathological condition.

Pathological states for which the methods of treatment of the invention are envisioned include a cancer, e.g., colorectal carcinoma, a prostate cancer a benign tumor, an immune disorder, an immune deficiency, an autoimmune disease, acquired immune deficiency syndrome, transplant rejection, allergy, an infection by a pathological organism or agent, an inflammatory disorder, arthritis, a hematopoietic disorder, a skin disorder, atherosclerosis, restenosis, a neurological disease, Alzheimer's disease, trauma, a surgical or traumatic wound, a spinal cord injury, and a skeletal disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a representation of the nucleotide (SEQ ID NO:1) and encoded amino acid sequence (SEQ ID NO:2) of clone 2820635.

FIG. 2 is a representation of the nucleotide (SEQ ID NO:3) and encoded amino acid sequence (SEQ ID NO:4) of clone 2826468.

FIG. 3 is a representation of the nucleotide (SEQ ID NO:5) and encoded amino acid sequence (SEQ ID NO:6) of clone 3186754.

FIG. 4 is a representation of the nucleotide (SEQ ID NO:7) and encoded amino acid sequence (SEQ ID NO:8) of clone 3277237.

FIG. 5 is a representation of the nucleotide (SEQ ID NO:9) and encoded amino acid sequence (SEQ ID NO:10) of clone 3277789.

FIG. 6 is a representation of the nucleotide (SEQ ID NO:11) and encoded amino acid sequence (SEQ ID NO:12) of clone 3293413.

FIG. 7 is a representation of the nucleotide (SEQ ID NO:13) and encoded amino acid sequence (SEQ ID NO:14) of clone 3470865.

FIG. 8 is a representation of the nucleotide (SEQ ID NO:15) and encoded amino acid sequence (SEQ ID NO:16) of clone 3473863.

FIG. 9 is a representation of the nucleotide (SEQ ID NO:17) and encoded amino acid sequence (SEQ ID NO:18) of clone 3487483.

FIG. 10 is a representation of the nucleotide (SEQ ID NO:19) and encoded amino acid sequence (SEQ ID NO:21) of clone 3492338.

FIG. 11A is a representation of the nucleotide (SEQ ID NO:11) and encoded amino acid sequence (SEQ IDNO:22) of clone 3540920-1.

FIG. 11B is a representation of the nucleotide (SEQ ID NO:140) and encoded amino acid sequence (SEQ IDNO:150) of clone 3540920-2.

FIG. 12A is a representation of the nucleotide (SEQ ID NO:23) and encoded amino acid sequence (SEQ ID NO:24) of clone 3885629-1.

FIG. 12B is a representation of the nucleotide (SEQ ID NO:151) and encoded amino acid sequence (SEQ ID NO:152) of clone 3885629-2.

FIG. 13 is a representation of the nucleotide (SEQ ID NO:25) and encoded amino acid sequence (SEQ ID NO:26) of clone 3886292.

FIG. 14 is a representation of the nucleotide (SEQ ID NO:27) and encoded amino acid sequence (SEQ ID NO:28) of clone 3903091.

FIG. 15 is a representation of the nucleotide (SEQ ID NO:29) and encoded amino acid sequence (SEQ ID NO:30) of clone 3906159.

FIG. 16A is a representation of the nucleotide (SEQ ID NO:31) and encoded amino acid sequence (SEQ ID NO:32) of clone 3921502-1.

FIG. 16B is a representation of the nucleotide (SEQ ID NO:153) and encoded amino acid sequence (SEQ ID NO:154) of clone 3921502-2.

FIG. 17 is a representation of the nucleotide (SEQ ID NO:33) and encoded amino acid sequence (SEQ ID NO:34) of clone 3923854.

FIG. 18 is a representation of the nucleotide (SEQ ID NO:35) and encoded amino acid sequence (SEQ ID NO:36) of clone 3923854.

FIG. 19A is a representation of the nucleotide (SEQ ID NO:37) and encoded amino acid sequence (SEQ ID NO:38) of clone 4002473-1.

FIG. 19B is a representation of the nucleotide (SEQ ID NO:155) and encoded amino acid sequence (SEQ ID NO:38) of clone 4002473-2.

FIG. 19C is a representation of the nucleotide (SEQ ID NO:154) and encoded amino acid sequence (SEQ ID NO:38) of clone 4002473-3.

FIG. 20 is a representation of the nucleotide (SEQ ID NO:39) and encoded amino acid sequence (SEQ ID NO:40) of clone 4031301.

FIG. 21 is a representation of the nucleotide (SEQ ID NO:41) and encoded amino acid sequence (SEQ ID NO:42) of clone 4030250.

FIG. 22 is a representation of the nucleotide (SEQ ID NO:43) and encoded amino acid sequence (SEQ ID NO:44) of clone 4160981.

FIG. 23A is a representation of the nucleotide (SEQ ID NO:45) and encoded amino acid sequence (SEQ ID NO:46) of clone 4192452-1.

FIG. 24 is a representation of SDS PAGE analysis of 2826468 protein secreted by SF9 cells.

FIG. 25 is a comparison of the relative expression of 2826468 sequences in various tissues.

FIG. 26A is a representation of SDS PAGE analysis of expression of 3122461 in 293 cells.

FIG. 26B is a representation of SDS PAGE analysis of expression and secretion of 312246 from SF9 cells.

FIG. 27 is a representation of SDS PAGE and silver staining analysis of 2 μg of purified GST-3122461 protein.

FIG. 28A is a representation of SDS PAGE analysis of expression of 3186754 in 293 cells.

FIG. 28B is a representation of SDS PAGE analysis of expression and secretion of 31886754 from SF9 cells.

FIG. 29 is a comparison of the relative expression of 3186754 sequences in various tissues.

FIG. 30 is a comparison of the relative expression of 3277237sequences in various tissues.

FIG. 31 is a representation of SDS PAGE and silver staining analysis of 1 μg of purified 3487483Ig protein.

FIG. 32 is a representation of 3487483 protein secreted by SF9 cells.

FIG. 33 is a comparison of the relative expression of 3487483 sequences in various tissues.

FIG. 34 is a comparison of the relative expression of 3492338 sequences in various tissues.

FIG. 35 is a comparison of the relative expression of 3540920 sequences in various tissues.

FIG. 36 is a comparison of the relative expression of 30903091 sequences in various tissues.

FIG. 37 is a comparison of the relative expression of 4030250 sequences in various tissues.

### DETAILED DESCRIPTION OF THE INVENTION

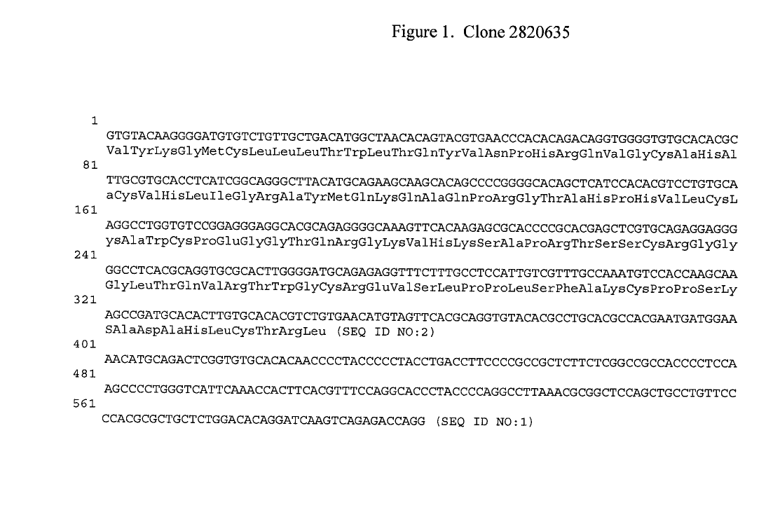

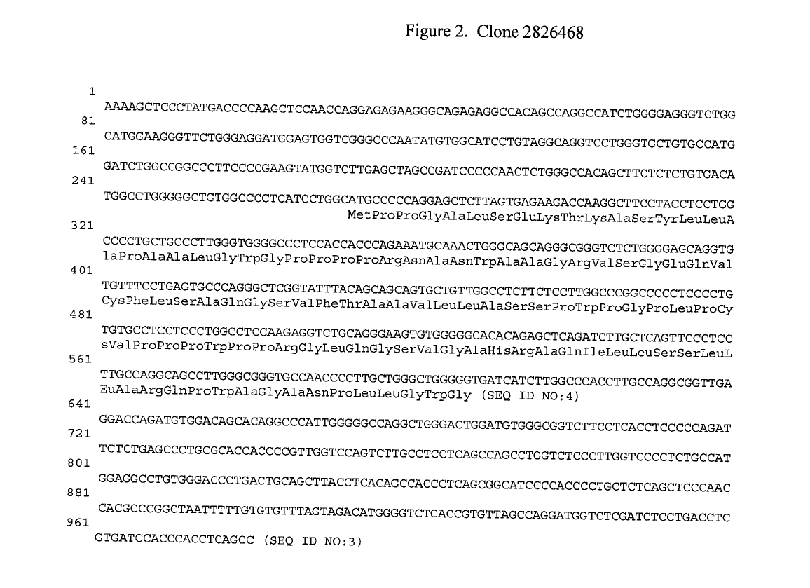

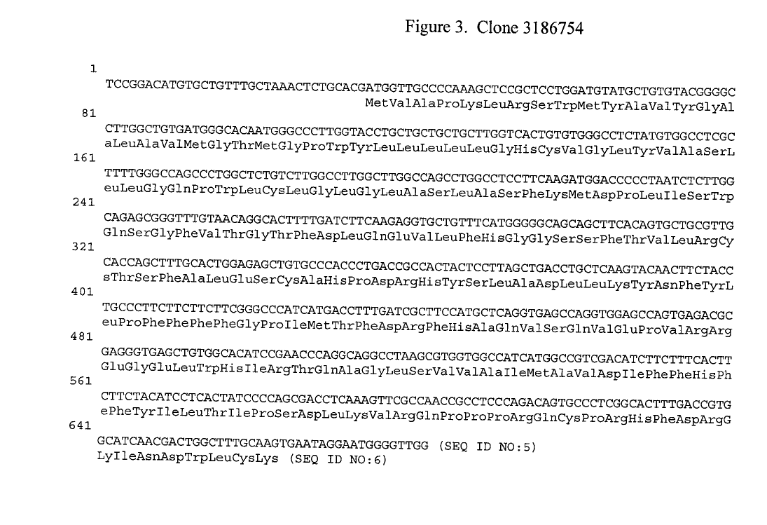

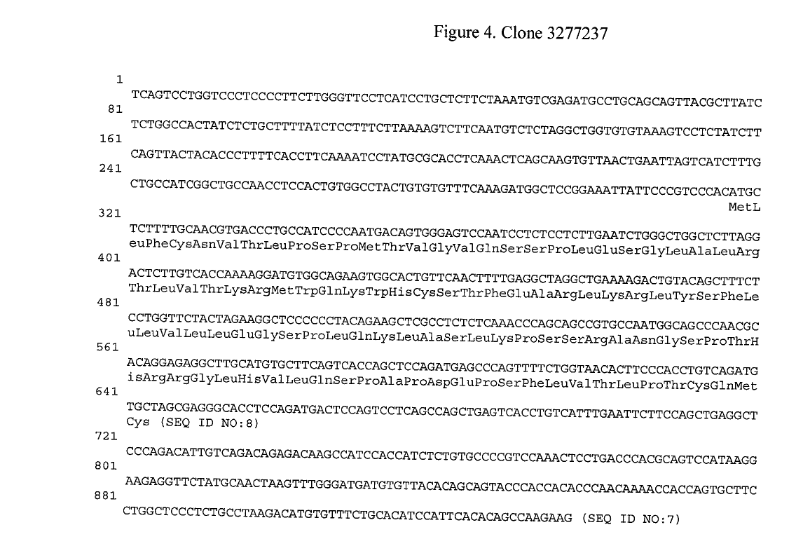

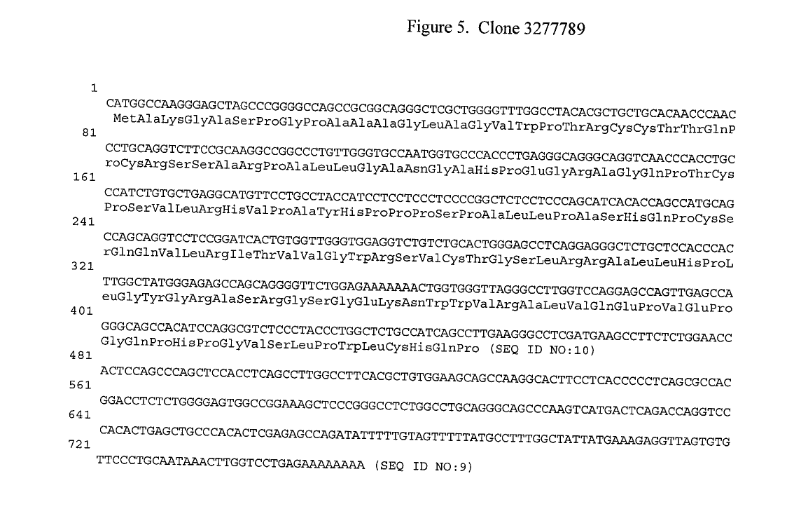

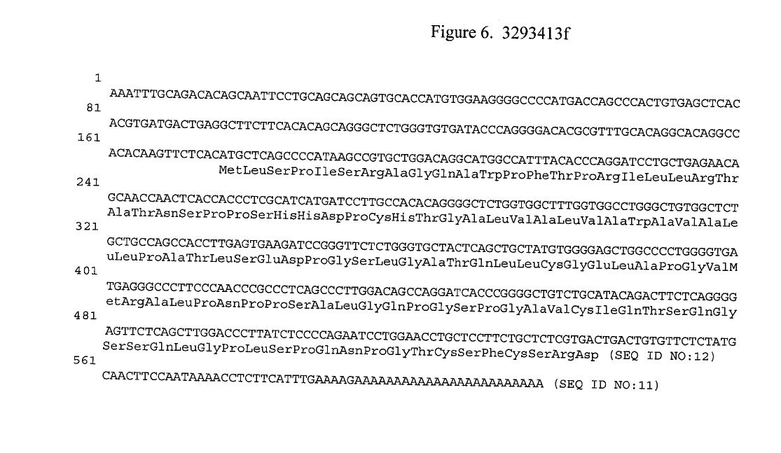

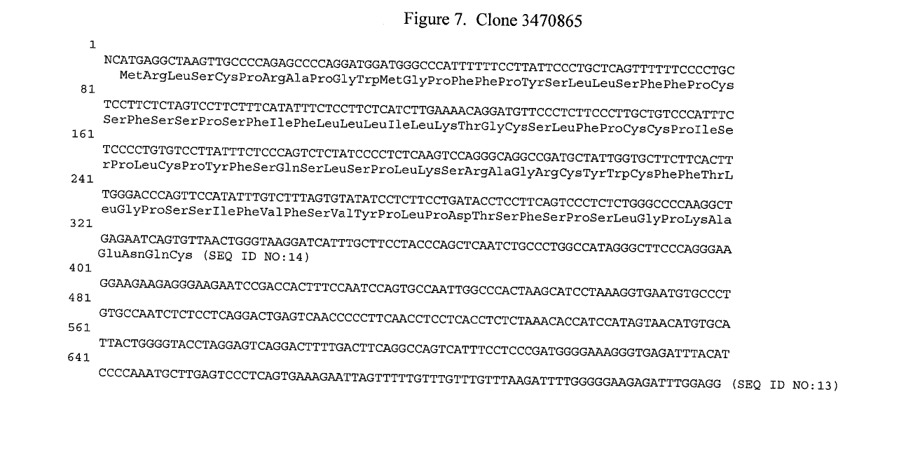

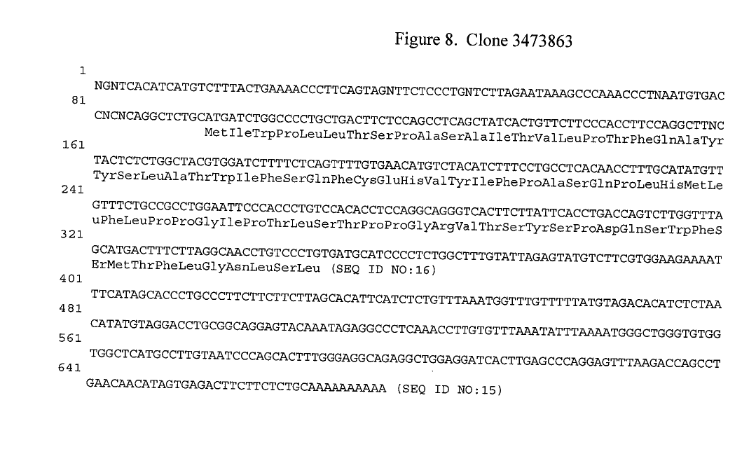

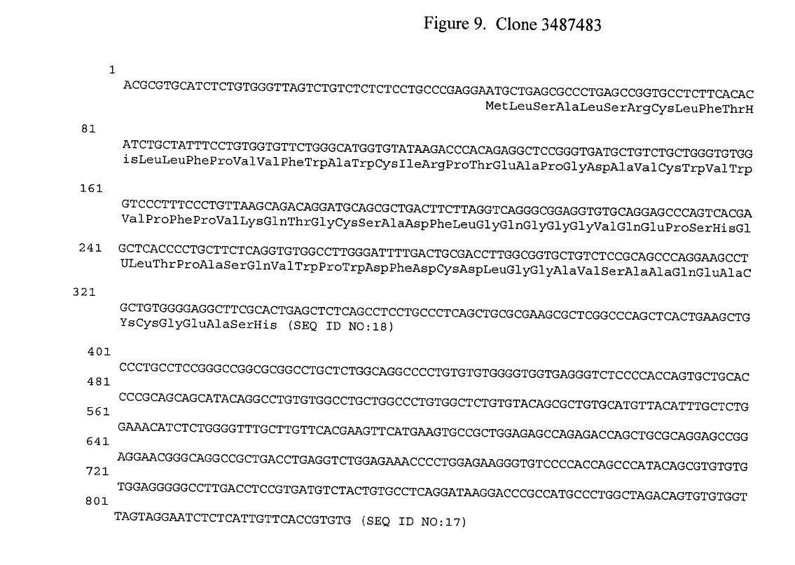

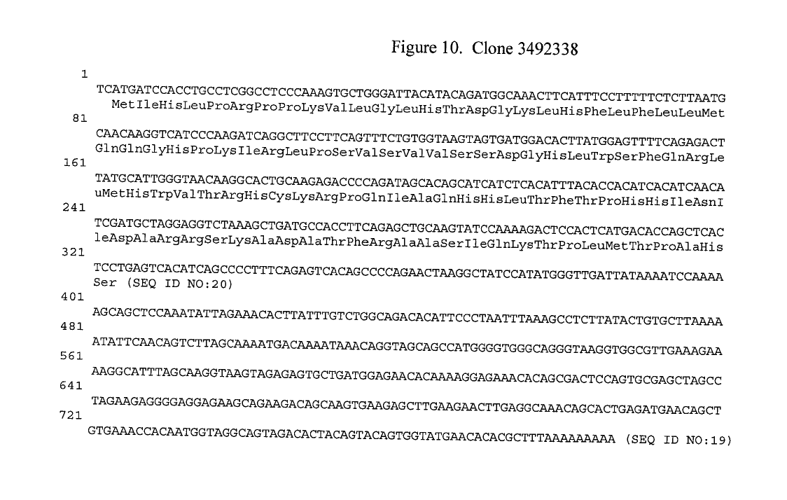

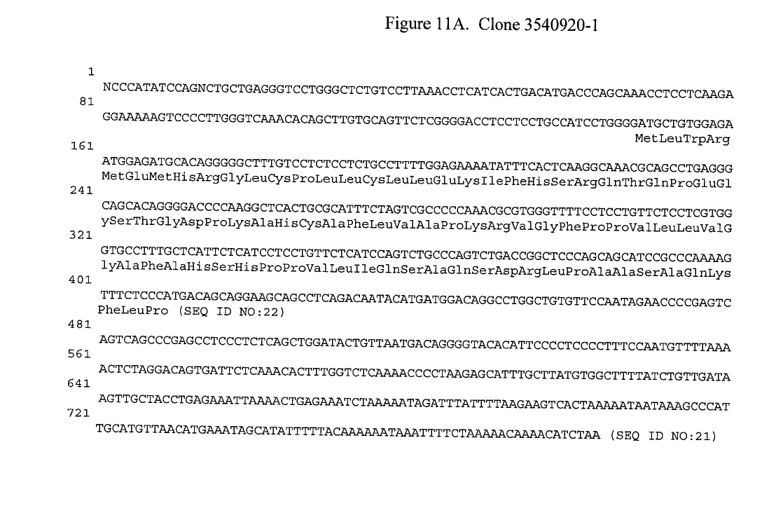

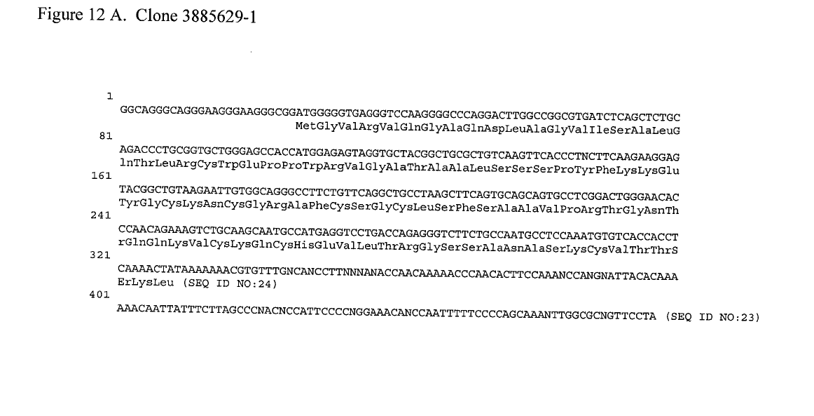

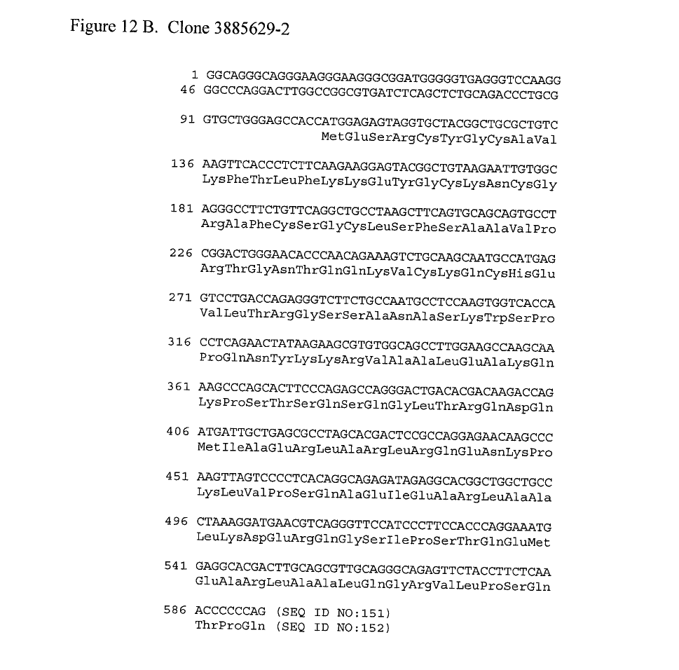

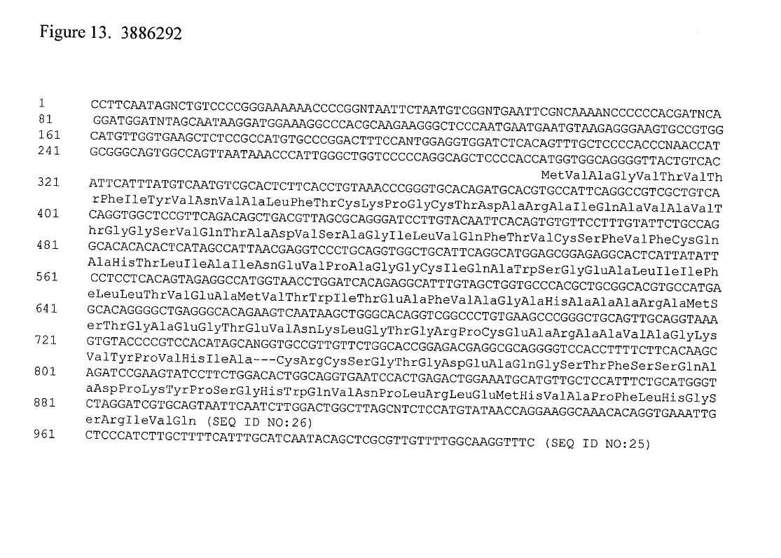

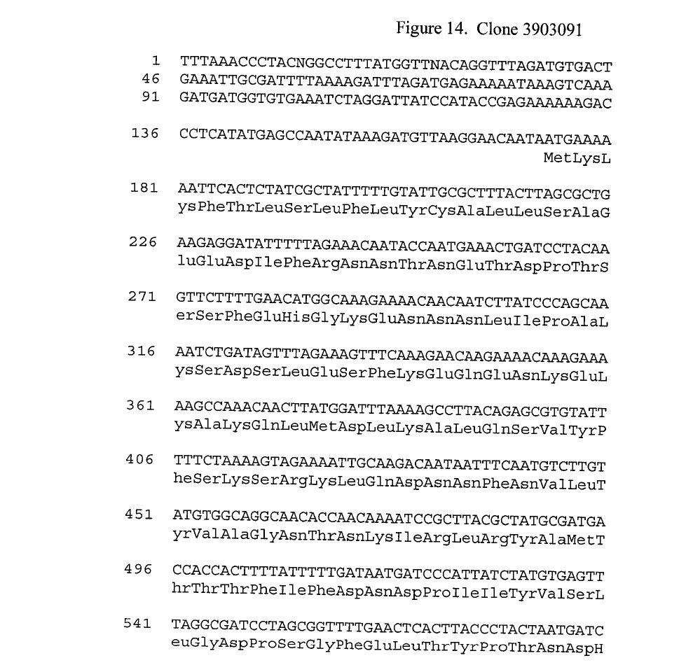

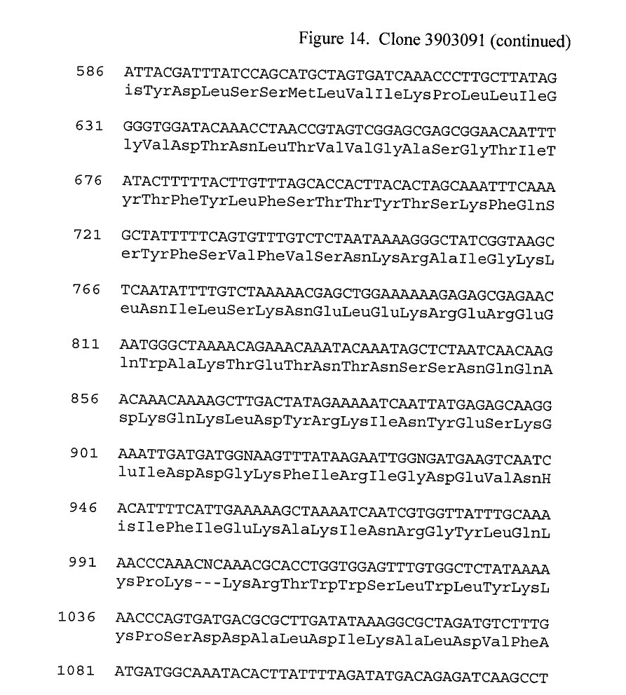

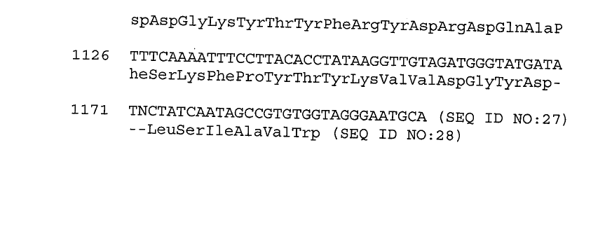

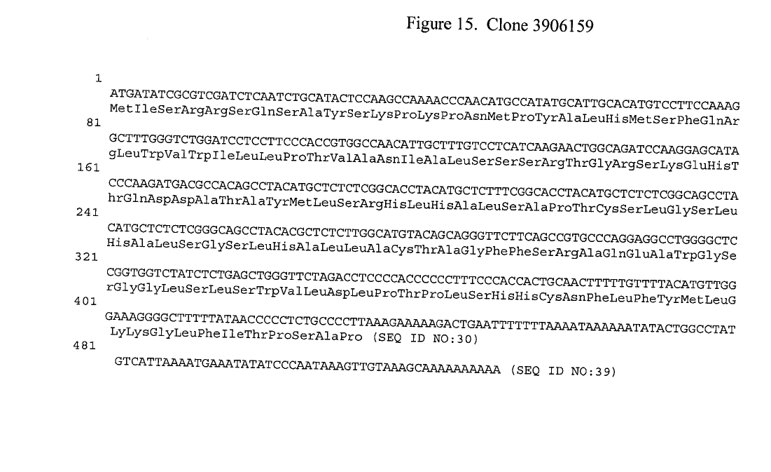

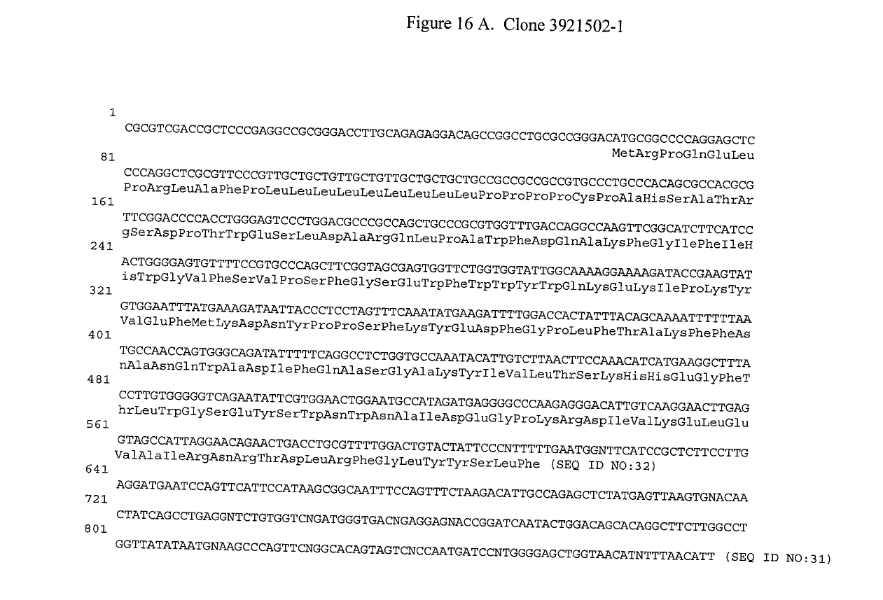

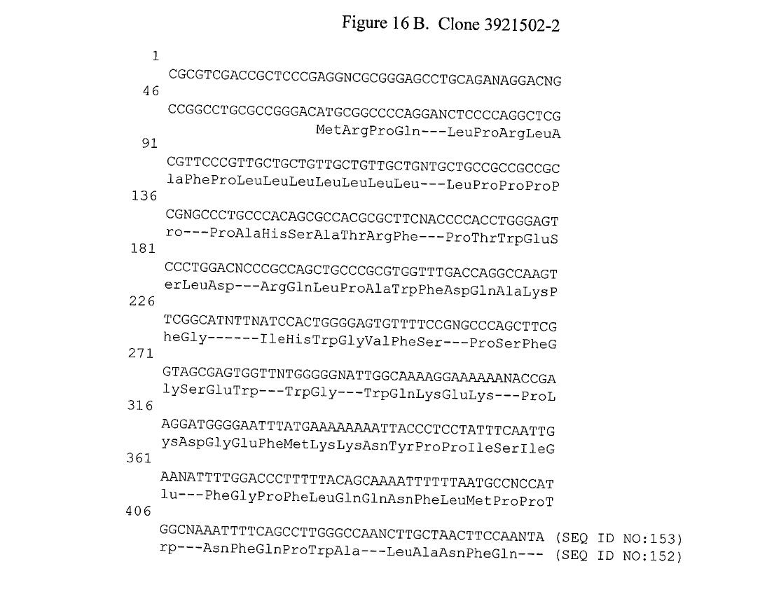

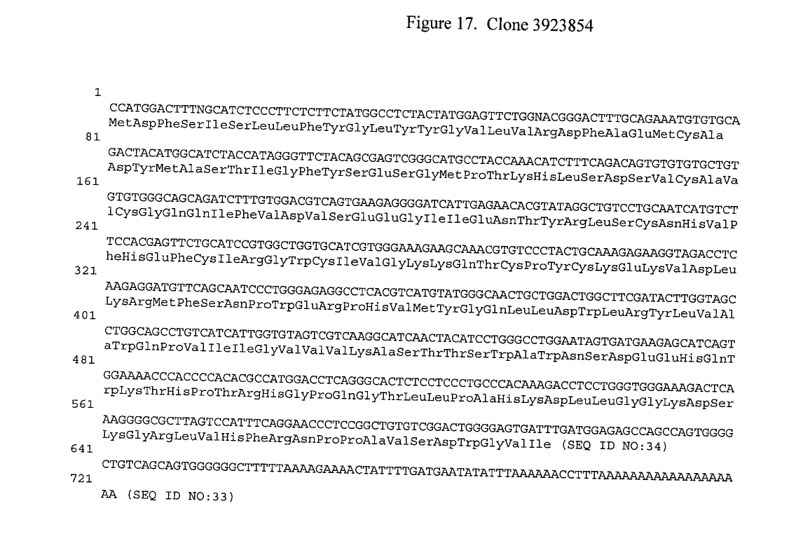

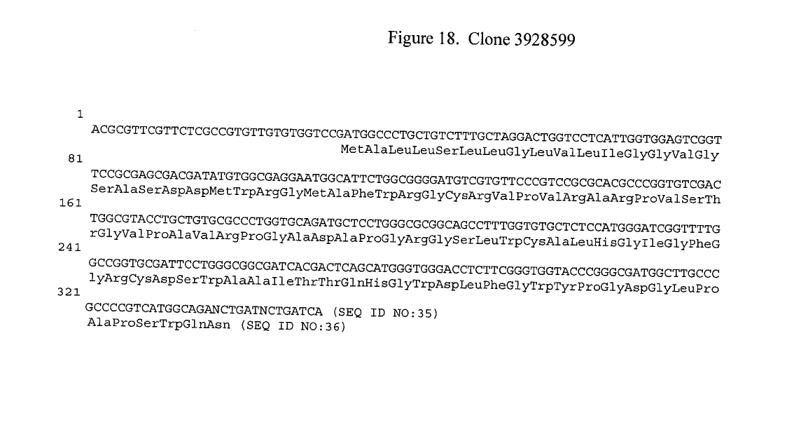

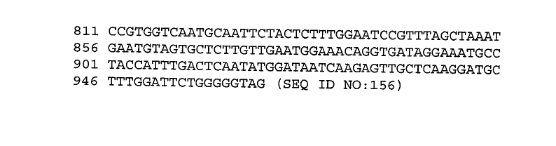

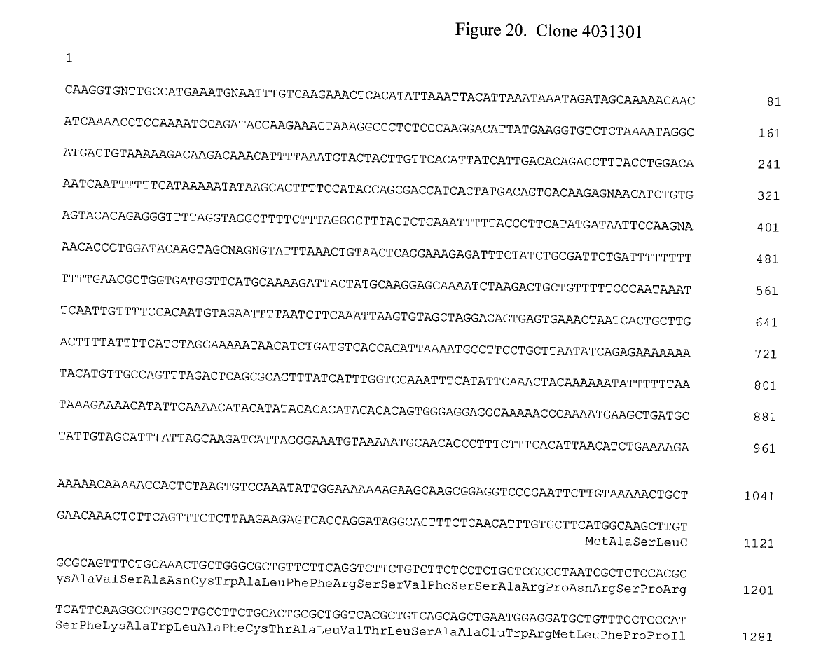

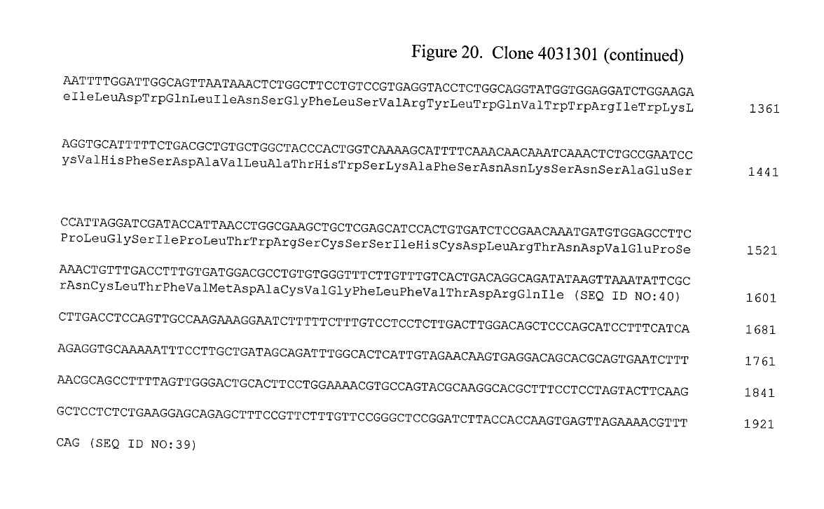

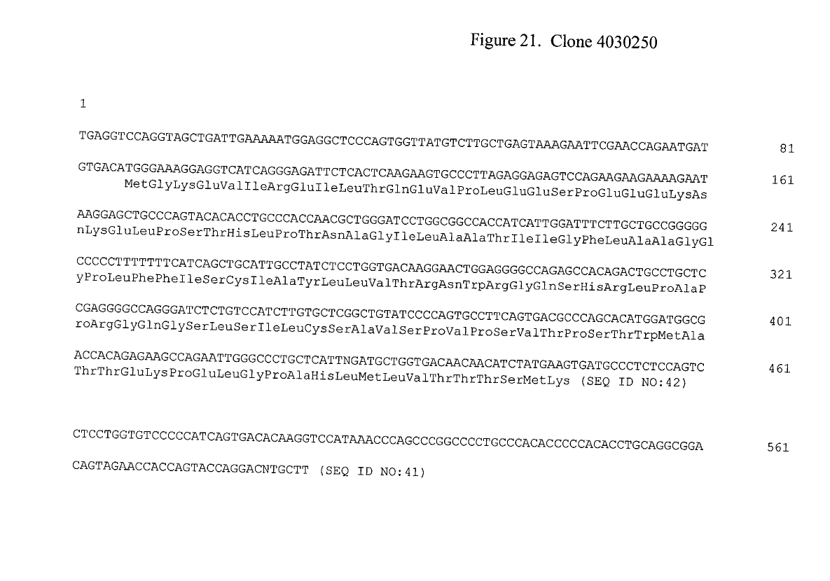

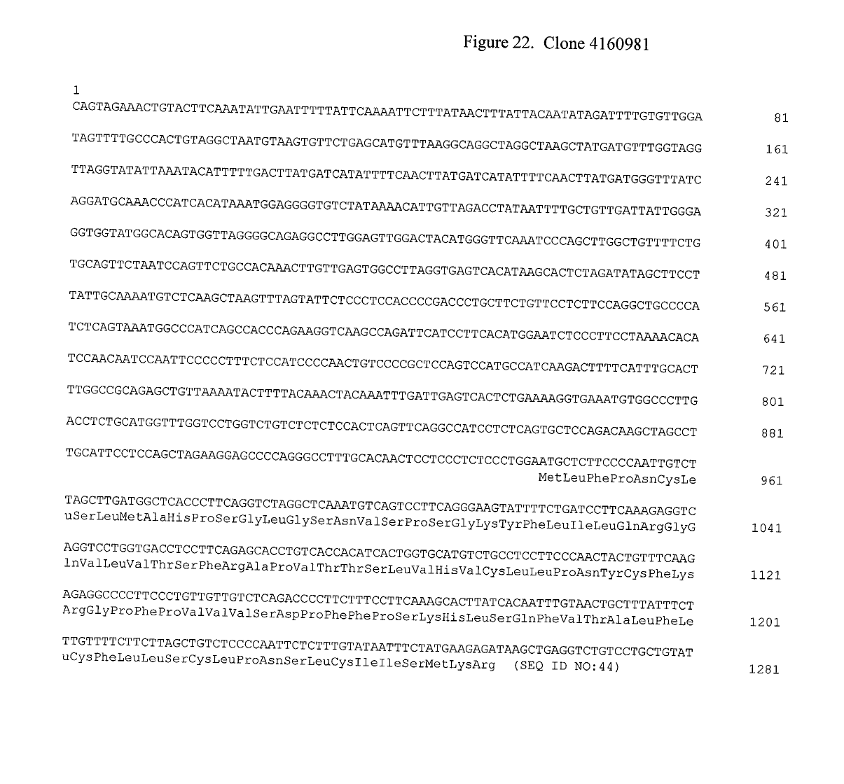

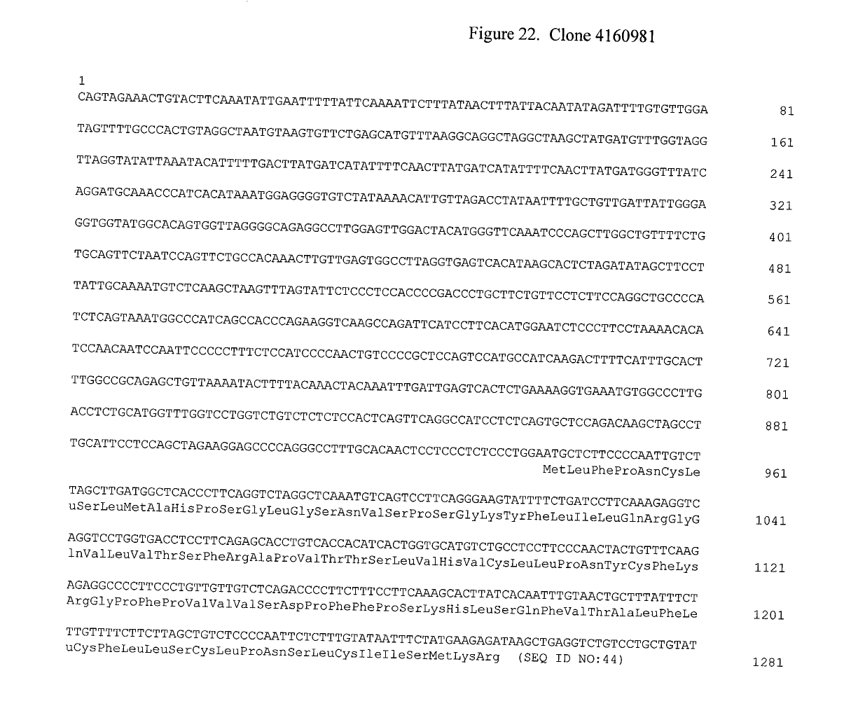

The invention is based in part on the discovery of 23 polypeptide sequences that contain sequence motifs that suggest the polypeptides are secreted or membrane bound proteins. Also disclosed are nucleic acids encoding these polypeptide sequences. These sequences are associated with the following clones and are disclosed in FIGS. 1-23. Amino acid residues indicated by dashes in the tables represent an unspecified amino acid.

The sequences and corresponding sequence identifier numbers assigned to the nucleotide sequences disclosed in FIGS. 1-23 are summarized in Table 1.

TABLE 1

| | | | Sequence Identifier |
|---|---|---|---|
| | | | Number of |
| | | Sequence Identifier Number | Encoded Amino |
| Clone | Table | of Nucleic Acid Sequence | Acid Sequence |
| 2820635 | 1 | SEQ ID NO: 1 | SEQ ID NO: 2 |
| 2826468 | 2 | SEQ ID NO: 3 | SEQ ID NO: 4 |
| 3186754 | 3 | SEQ ID NO: 5 | SEQ ID NO: 6 |
| 3277237 | 4 | SEQ ID NO: 7 | SEQ ID NO: 8 |
| 3277789 | 5 | SEQ ID NO: 9 | SEQ ID NO: 10 |
| 3293413 | 6 | SEQ ID NO: 11 | SEQ ID NO: 12 |
| 3470865 | 7 | SEQ ID NO: 13 | SEQ ID NO: 14 |
| 3473863 | 8 | SEQ ID NO: 15 | SEQ ID NO: 16 |
| 3487483 | 9 | SEQ ID NO: 17 | SEQ ID NO: 18 |
| 3492338 | 10 | SEQ ID NO: 19 | SEQ ID NO: 20 |
| 3540920-1 | 11A | SEQ ID NO: 21 | SEQ ID NO: 22 |
| 3885629-1 | 12A | SEQ ID NO: 23 | SEQ ID NO: 24 |
| 3886292 | 13 | SEQ ID NO: 25 | SEQ ID NO: 26 |
| 3903091 | 14 | SEQ ID NO: 27 | SEQ ID NO: 28 |
| 3906159 | 15 | SEQ ID NO: 29 | SEQ ID NO: 30 |
| 3921502-1 | 16A | SEQ ID NO: 31 | SEQ ID NO: 32 |
| 3923854 | 17 | SEQ ID NO: 33 | SEQ ID NO: 34 |
| 3928599 | 18 | SEQ ID NO: 35 | SEQ ID NO: 36 |
| 4002473-1 | 19A | SEQ ID NO: 37 | SEQ ID NO: 38 |
| 4031301 | 20 | SEQ ID NO: 39 | SEQ ID NO: 40 |
| 4030250 | 21 | SEQ ID NO: 41 | SEQ ID NO: 42 |
| 4160981 | 22 | SEQ ID NO: 43 | SEQ ID NO: 44 |
| 4192452-1 | 23A | SEQ ID NO: 45 | SEQ ID NO: 46 |
| 3540920-2 | 11B | SEQ ID NO: 149 | SEQ ID NO: 150 |
| 3885629-2 | 12B | SEQ ID NO: 151 | SEQ ID NO: 152 |
| 3921502-2 | 16B | SEQ ID NO: 153 | SEQ ID NO: 154 |
| 4002473-2 | 19B | SEQ ID NO: 155 | |
| 4002473-3 | 19C | SEQ ID NO: 156 | |

Unless stated otherwise, the term “SECX nucleic acid” or “SECX encoding nucleic acid” is understood to refer to any nucleic acid having a sequence corresponding to the nucleic acid sequence identifier number shown in Table 1. Thus, a SECX nucleic acid can be a nucleic acid sequence which includes any of SEQ ID NOs: 1, 3, 5, 7, 9, 11, 13, 15, 17, 19, 21, 23, 25, 27, 29, 31, 33, 35, 37, 39, 41, 43, 45, 47, 149, 151, 153, 155,or 156.

Unless stated otherwise, the term “SECX polypeptide” or “SECX protein” is understood to refer to any polypeptide having a sequence corresponding to the amino acid identifier number shown in Table 1. Thus, a SECX polypeptide sequence can be a polypeptide which includes any of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154.

Below follows descriptions of the novel secreted proteins disclosed herein.

Clone 2820635

The nucleic acid provided by clone 2820635 is 508 nucleotides in length and includes an open reading frame encoding a secreted protein having 116 residues (also referred to herein as “2820635 protein”) from nucleotides 1 to 348 (SEQ ID NO:147). The sequence lacks an initiation codon, suggesting that it may be a 3′ fragment (i.e., a C-terminal polypeptide fragment) of a larger protein.

A putative signal peptide cleavage site occurs between residues 18 and 19 of the encoded polypeptide. Thus, the 2820635 polypeptide includes a signal sequence region having amino acids 1-18 (SEQ ID NO:90) and amino acids 19-116 (SEQ ID NO:91), which corresponds to the polypeptide lacking the signal sequence region.

Clone 2826468

The nucleic acid provided by clone 2826468 is obtained from bone marrow 5PH16 nucleic acids. It is 980 nucleotides in length and includes an open reading frame encoding a secreted protein of 112 residues (also referred to herein as “2826468 protein”) from nucleotides 272 to 607 (SEQ ID NO:92). No signal peptide cleavage site was identified in the amino acid sequence.

The amino acid sequence of 2826468 protein was searched against the GenBank database using the BLASTP search protocol. The search identified a similarity to human deltex protein. The predicted encoded protein has a high probability of localization in the plasma membrane and/or the endoplasmic reticulum.

Transcripts corresponding to clone 2826468 nucleic acid sequences are found in the cerebellum and hippocampus regions of brain. They are also found in colon cancer cells at high levels.

Clone 3186754

The nucleic acid provided by clone 3186754 is 682 nucleotides in length and includes an open reading frame encoding a secreted protein of 210 residues (also referred to herein as “3186754 protein”) from nucleotides 34 to 663 (SEQ ID NO:148).

The amino acid sequence of 3186754 protein shares some sequence similarity to a hypothetical protein in the Saccharomyces cerevisiae Mad1-Scy1 intergenic region. The protein also shares similarity to human amyloid lambda light chain variable region. A presumptive signal peptide cleavage site occurs between positions 61 and 62. Thus, the 3186754 polypeptide includes a signal sequence region having amino acids 1-61 (SEQ ID NO:93) and amino acids 62-210 (SEQ ID NO:94), which corresponds to the polypeptide lacking the signal sequence region.

Transcripts corresponding to clone 3186754 nucleic acid sequences are found in the heart, skeletal muscle, normal brain, and spinal cord tissue. Only weak expression is detected in tumor cells.

Clone 3277237

The nucleic acid provided by clone 3277237 is 937 nucleotides in length and includes an open reading frame encoding a secreted protein of 109 residues (also referred to herein as “3277237 protein”) from nucleotides 317 to 643 (SEQ ID NO:95).

The amino acid sequence of 2353875 protein was searched against the GenBank database using the BLASTP search protocol. No similarity to any protein having a high probability for a match was found. The predicted encoded protein has high probabilities of localization in the plasma membrane and/or the endoplasmic reticulum.

Transcripts corresponding to clone 3277237 nucleic acid sequences are found at high levels in fetal and adult brain, with strong expression seen in cerebellum and hippocampus. Moderate expresssion is also observed in the heart and thymus, and in the lung cancer tumor cell line HCI-H522.

Clone 3277789

The nucleic acid provided by clone 3277789 is 754 nucleotides in length and includes an open reading frame encoding a secreted protein of 149 residues (also referred to herein as “3277789 protein”) from nucleotides 2 to 448 (SEQ ID NO:96). No signal sequence is predicted in the amino acid sequence.

The amino acid sequence of 3277789 protein was compared to known sequences using the GenBank BLASTP search protocol. The BLASTP search shows moderate similarity to human mucin. The sequence of the predicted encoded protein indicates it has a high probability of secretion through the endoplasmic reticulum and/or the lysosomal membrane.

Clone 3293413f

As used herein, this clone is also referred to as 3293413. The nucleic acid provided by clone 3293413 is 616 nucleotides in length and includes an open reading frame encoding a secreted protein of 123 amino acids (also referred to herein as “3293413 protein”) from nucleotides 175 to 543 (SEQ ID NO:97).

The amino acid sequence of 3293413 protein was searched against the GenBank database using the BLASTP search protocol. The search indicates that the 3293413 protein shows similarity to the human bullous pemphigoid antigen-2, which is a collagen The predicted encoded protein has high probabilities of secretion through the plasma membrane and/or the inner mitochondrial membrane. A presumptive signal peptide cleavage site is found between positions 55 and 56. Thus, the 3293413f polypeptide includes a signal sequence region having amino acids 1-55 (SEQ ID NO:98) and amino acids 56-123 (SEQ ID NO:99), which corresponds to a polypeptide lacking the signal sequence region.

Clone 3470865

The nucleic acid provided by clone 3470865 is 719 nucleotides in length and includes an open reading frame encoding a secreted protein of 110 residues (also referred to herein as “3470865 protein”) from nucleotides 3 to 332 (SEQ ID NO:100).

The amino acid sequence of 3470865 protein was searched against the GenBank database using the BLASTP search protocol. The search identified a similarity to a hypothetical ORF-8 protein in Leishmania tarentolae mitochondria. The predicted encoded protein has a high probability of secretion through the plasma membrane. A presumptive signal peptide cleavage site is found between positions 46 and 47. Thus, the 347085 polypeptide includes a signal sequence region having amino acids 1-46 (SEQ ID NO:101) and amino acids 47-110 (SEQ ID NO:102), which corresponds to the polypeptide lacking the signal sequence region.

Clone 3473863

The nucleic acid provided by clone 3473863 is 678 nucleotides in length and includes an open reading frame encoding a secreted protein of 85 residues (also referred to herein as “3473863 protein”) from nucleotides 95 to 349 (SEQ ID NO:103).

The amino acid sequence of 3473863 protein was searched against the GenBank database using the BLASTP search protocol. The search identified a similarity to rex protein from simian T-cell lymphotropic virus type 2, as well as a weak similarity to human RFPL1S protein. The predicted encoded protein may be secreted through the plasma membrane or the peroxisomal membrane. A presumptive signal peptide cleavage site is found between positions 21 and 22. Thus, the 3473863 protein includes a signal sequence region having amino acids 1-21 (SEQ ID NO:104) and amino acids 22-85 (SEQ ID NO:105), which corresponds to the polypeptide lacking the signal sequence region.

Clone 3487483

The nucleic acid provided by clone 3487483 is 830 nucleotides in length and includes an open reading frame encoding a secreted protein of 98 residues (also referred to herein as “3487483protein”) from nucleotides 47 to 340 (SEQ ID NO:106).

The amino acid sequence of 3487483 protein was searched against the GenBank database using the BLASTP search protocol. The search identified a similarity to a portion of a hypothetical 25.7 kDa protein from Synechocystis sp. (strain PCC 6803), and to a portion of human immunoglobulin heavy chain precursor. The predicted encoded protein may be secreted extracellularly. Presumptive signal peptide cleavage sites are found between positions 23 and 24, or between residues 29 and 30. Thus, in one embodiment, the 3487483 protein includes a signal sequence region having amino acids 1-23 (SEQ ID NO:107) and a polypeptide having amino acid sequences 24-98 (SEQ ID NO:108), which lacks the amino acid 1-23 signal sequence region. In another embodiment, the 3487483 protein includes a signal sequence region having amino acids 1-29 (SEQ ID NO:109) and a polypeptide having the amino acid sequences 30-98 (SEQ ID NO:110), which lacks the amino acid 1-28 signal sequence region.

The 3487483 gene is expressed in many different tissues. It is highly expressed in lung cancer cell lines SHP-77 and NCI-H460, and prostate cancer cell line PC3.

Clone 3492338

The nucleic acid provided by clone 3492338 is 787 nucleotides in length and includes an open reading frame encoding a secreted protein having 107 residues (also referred to herein as “3492338protein”) from nucleotides 3 to 323 (SEQ ID NO:111). No signal peptide cleavage site was identified in the 3492338 amino acid sequence.

The amino acid sequence of 3492338 protein was searched against the GenBank database using the BLASTP search protocol. The search identified a similarity to a portion of enterocin I from Enterococcus faecium, and to a portion of human sorbitol dehydrogenase (EC 1.1.1.14).

Clones 3540920-1 and 3540920-2

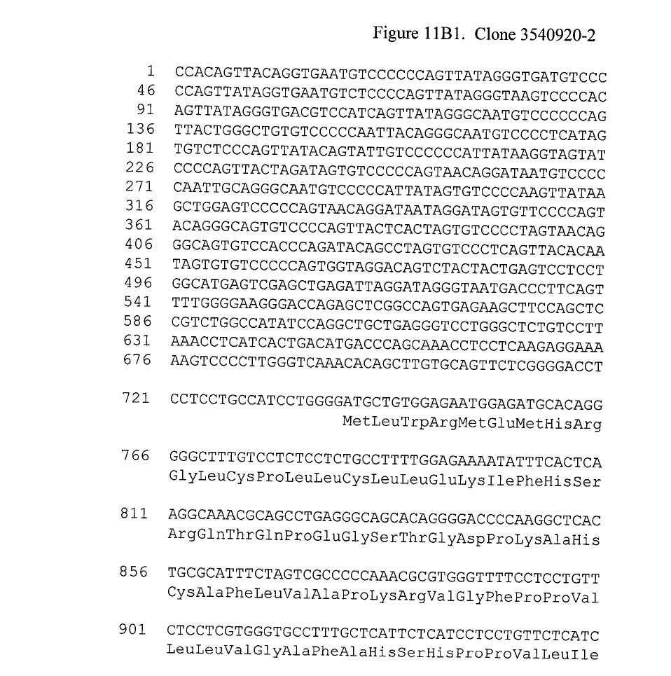

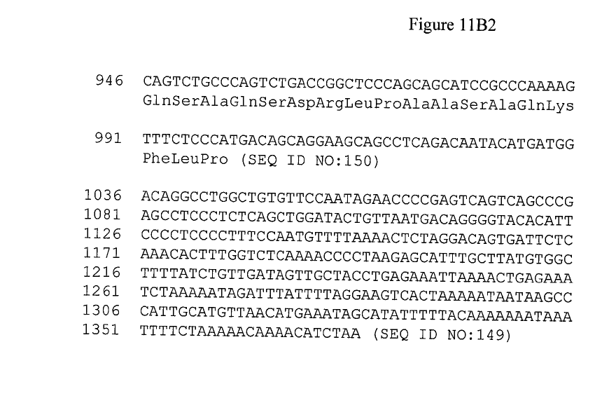

Two clones were obtained which both contain the same open reading frame. These are shown in FIGS. 11A and 11B. The nucleic acid provided by clone 3540920-1 is 784 nucleotides in length and includes an open reading frame encoding a secreted protein 87 residues in length (also referred to herein as “3540920protein”) from nucleotides 149 to 409 (SEQ ID NO:112). The nucleic acid given by clone 3540920-2 is 1373 nucleotides long, and overlaps the sequence of 3540920-1 beginning at base 591. As noted above, the open reading frames are the same. Otherwise. there are ambiguities where 3540920-1 has an unidentified nucleotide present, and there are four gaps or mismatches in the 3′ untranslated region.

The amino acid sequence of 3540920 protein was searched against the GenBank database using the BLASTP search protocol. The search identified no similarity to any known human protein. The predicted encoded protein has a moderate possibility of being secreted through the membrane of the endoplasmic reticulum. A presumptive signal peptide cleavage site is found between positions 27 and 28. Thus, the 3540920 polypeptide includes a signal sequence region having amino acids 1-27 (SEQ ID NO:113) and amino acids 28-87 (SEQ ID NO:114), which corresponds to the polypeptide lacking the signal sequence region.

Expression of 3540920 sequences is detected in many different tissues, with strongest expression seen in the brain (cerebellum). Over-expression is also observed in three lung cancer cell lines (SHP-77, NCI-H460 and NCI-H522) relative to normal lung tissue and in one prostate cell line (PC3) relative to normal prostate tissue.

Clone 3885629-1

The nucleic acid provided by clone 3885629-1 is 477 nucleotides in length and includes an open reading frame encoding a secreted protein of 101 residues (also referred to herein as “3885629-1 protein”) from nucleotides 26 to 328 (SEQ ID NO:115). No signal peptide cleavage site has been identified in the amino acid sequence of the 3885639 protein.

The amino acid sequence of 3885629 protein was searched against the GenBank database using the BLASTP search protocol. The search identified a similarity to a putative vacuolar protein sorting associated protein of Schizosaccharomyces pombe, as well as to a short portion of a human complete coding sequence (SPTREMBL ACC:O14964). The predicted encoded protein has a moderate possibility of being secreted through the membrane of microbodies (peroxisomes).

Clone 3885629-2

The nucleic acid provided by clone 3885629-2 is 594 nucleotides in length (SEQ ID NO:151) and includes an open reading frame encoding a protein fragment of 163 residues (also referred to herein as “3885629-2 protein”) from nucleotides 116 to 594 (SEQ ID NO:152). In a BLASTP search against public databases, the protein fragment was found to bear no close similarity to any known protein.

Clone 3886292

The nucleic acid provided by clone 3886292 is 1017 nucleotides in length and includes an open reading frame encoding a secreted protein having 241 residues (also referred to herein as “3886292protein”) from nucleotides 172 to 894 (SEQ ID NO:116).

The amino acid sequence of 3886292 protein was searched against the GenBank database using the BLASTP search protocol. The search identified no similarity to any known protein. The predicted encoded protein has a good probability of being secreted through the plasma membrane and/or the Golgi membrane. A presumptive signal peptide cleavage site is found between positions 67 and 68. Thus, the 3886292 protein includes a signal peptide region having amino acids 1-67 (SEQ ID NO:117) and a polypeptide having the amino acid sequence 68-241 (SEQ ID NO:118), which lacks the signal peptide region.

Clone 3903091

The nucleic acid provided by clone 3903091 is 1201 nucleotides in length and includes an open reading frame encoding a secreted protein having 339 residues (also referred to herein as “3903091protein”) from nucleotides 174 to 1190 (SEQ ID NO:119). A presumptive signal peptide cleavage site is found between amino acids 17 and 18. Thus, the 3903091 protein includes a signal peptide region having amino acids 1-17 (SEQ ID NO:120) and a polypeptide having the amino acid sequence 18-339 (SEQ ID NO:121), which lacks the signal peptide region.

The amino acid sequence of 3903091 protein was searched against the GenBank database using the BLASTP search protocol. The search identified a fragment of the 3903091 protein having a high similarity to a putative protein of Helicobacter pylori J99.

The 3903091 gene is expressed in a variety of locations in the brain, including the amygdala, cerebellum, hippocampus, hypothalamus, thalamus and substantia nigra. Overexpression is also observed in colon cancer cell line HCT-116 relative to normal colon tissue, in two lung cancer cell lines (SHP-77 and NCI-H460) relative to normal lung tissue, in one prostate cancer line (PC3) relative to normal prostate tissue, and in one melanoma (LOX IMVI).

Clone 3906159

The nucleic acid provided by clone 3906159 is 529 nucleotides in length and includes an open reading frame encoding a secreted protein having 241 residues (also referred to herein as “3906159protein”) from nucleotides 1 to 432 (SEQ ID NO:122). A presumptive signal peptide cleavage site is found between residues 43 and 44. Thus, the 3906159 protein includes a signal peptide region having amino acids 1-43 (SEQ ID NO:123) and a polypeptide having the amino acid sequence 44-241 (SEQ ID NO:124), which lacks the signal peptide region.

The amino acid sequence of the 3906159 protein was searched against the GenBank database using the BLASTP search protocol. The search identified a fragment of the 3906159 protein having a moderate similarity to a fragment of enoyl-CoA hydratase from Arabidopsis thaliana. No similarity to a human protein was identified. The predicted encoded protein has a high probability of being secreted through the microbody (peroxisomal) membrane and/or the plasma membrane.

Clone 3921502-1

The nucleic acid provided by clone 3921502-1 is 876 nucleotides in length and includes an open reading frame encoding a secreted protein having 184 residues from nucleotides 63 to 614 (SEQ ID NO:125). A presumptive signal peptide cleavage site occurs between residues 28 and 29. Thus, the 3921502-1 protein includes the signal peptide region having amino acid sequences 1-28 (SEQ ID NO:126) and a polypeptide having amino acid sequences 29-184 (SEQ ID NO:127), which lacks the signal peptide region.

The amino acid sequence of 3921502-1 protein was searched against the GenBank database using the BLASTP search protocol. The search identified a high degree of similarity to a fragment of human tissue alpha-L-fucosidase precursor (EC 3.2.1.51), which has 461 residues. The predicted encoded protein has a high probability of being secreted through the plasma membrane.

Clone 3921502-2

The nucleic acid provided by clone 3921502-2 is 449 nucleotides in length (SEQ ID NO:153) and includes an open reading frame from nucleotides 63 to 449encoding a protein fragment having 129 residues (SEQ ID NO:154).

The nucleotide sequence of 3921502-2 protein was searched against the GenBank database using the BLASTN search protocol. The search identified a high degree of similarity to a gene for alpha-L-fucosidase precursor (EC 3.2.1.51) from humans and from dogs.

Clone 3923854

The nucleic acid provided by clone 3923854 is 722 nucleotides in length and includes an open reading frame encoding a secreted protein having 205 residues (also referred to herein as “3923854protein”) from nucleotides 3 to 617 (SEQ ID NO:128). A presumptive signal peptide cleavage site occurs between residues 22 and 23. Thus, the 3923854 protein includes the signal peptide region having amino acid sequences 1-22 (SEQ ID NO:129) and a polypeptide having amino acid sequences 23-205 (SEQ ID NO:130), which lacks the signal peptide region.

The amino acid sequence of 3923854 protein was searched against the GenBank database using the BLASTP search protocol. The search identified a moderate degree of similarity to a fragment of a hypothetical 47.6 kKa protein C16C10.5 in chromosome III of Caenorahabditis elegans, as well as to a fragment of human protein R33683-3. The predicted encoded protein has a moderate probability of being secreted through the plasma membrane.

Clone 3928599

The nucleic acid provided by clone 3928599 is 350 nucleotides in length and includes an open reading frame encoding a secreted protein having 102 residues (also referred to herein as “3928599protein”) from nucleotides 33 to 339 (SEQ ID NO:131). A signal peptide cleavage site is predicted between residues 18 and 19. Thus, the 3928599 protein includes a signal peptide region 1-18 (SEQ ID NO:132) and a polypeptide having the amino acid sequences 19-102 (SEQ ID NO:133), which lacks the signal peptide region.

The amino acid sequence of 3928599 protein was searched against the GenBank database using the BLASTP search protocol. The search identified a moderate degree of similarity to a fragment of bovine endocnuclease G precursor (EC 3.1.30.-). The predicted encoded protein has a high probability of being secreted through the plasma membrane.

Clones 4002473-1, 4002473-2, and 4002473-3

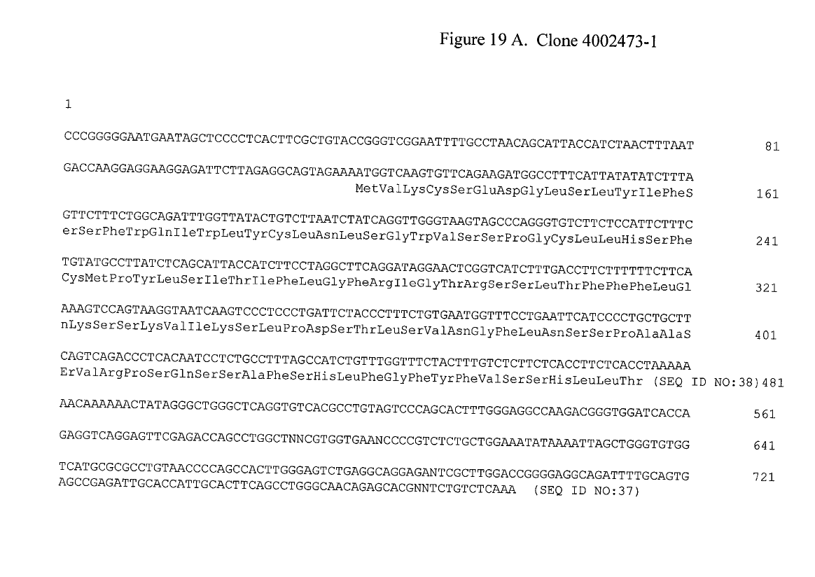

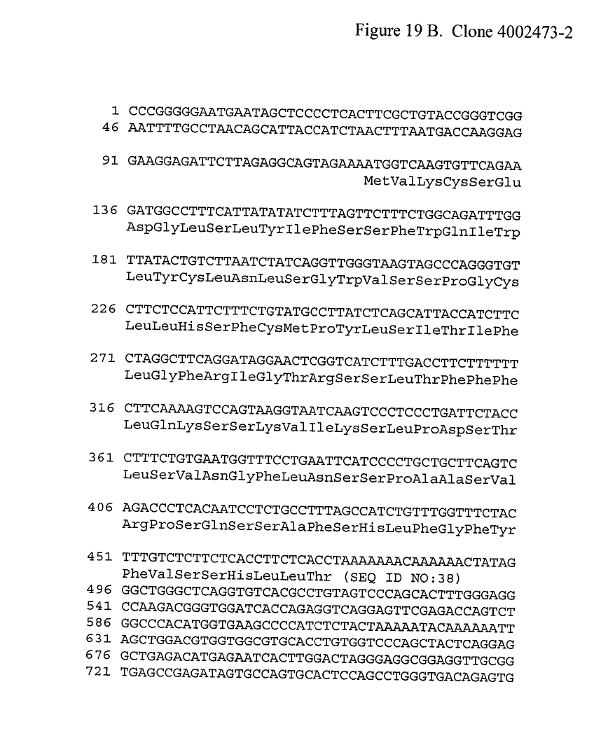

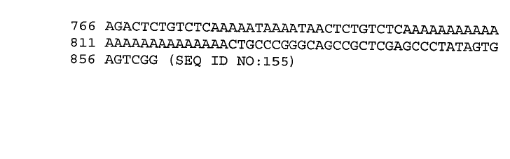

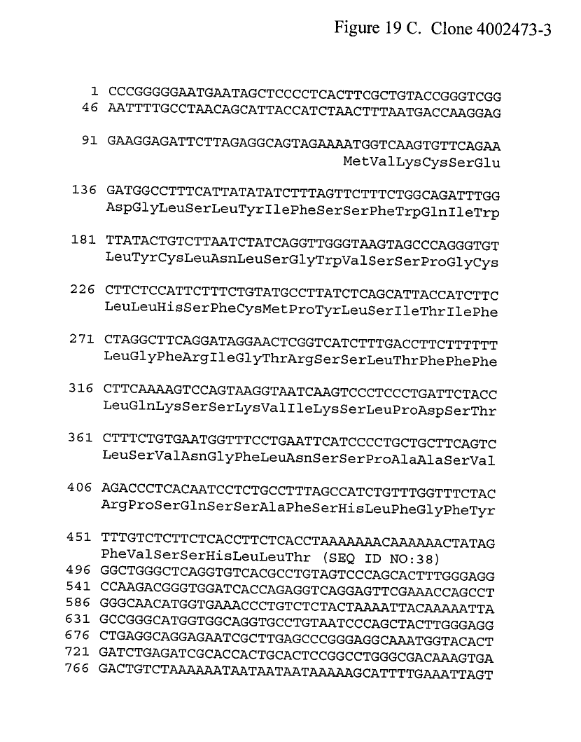

Three nucleic acids differing in the 3′ untranslated region are given clones 4002473-1, 4002473-2, and 4002473-3 (FIGS. 19A, 19B and 19C; SEQ ID NOs: 37, 155 and 156). Each one includes an open reading frame encoding a secreted protein having 119 residues (also referred to herein as “4002473 protein”) from nucleotides 118 to 474 (SEQ ID NO:134). A signal peptide cleavage site is predicted between residues 32 and 33. Thus, the 4002473 protein includes a signal peptide region 1-32 (SEQ ID NO:135) and a polypeptide having the amino acid sequence 33-119 (SEQ ID NO:136), which lacks the signal peptide region.

The amino acid sequence of 4002473 protein was searched against the GenBank database using the BLASTP search protocol. The search identified a low degree of similarity to a fragment of the protein EATRO 164 kinetoplast CR3 from Trypanosoma brucei brucei. The predicted encoded protein has a moderate probability of being secreted through the plasma membrane and/or the microbody (peroxisomal) membrane.

Clone 4031301

The nucleic acid provided by clone 4031301 is nucleotides in length and includes an open reading frame encoding a secreted protein having 159 residues (also referred to herein as “4031301protein”) from nucleotides 1108 to 1584 (SEQ ID NO:137). A signal peptide cleavage site is predicted between residues 48 and 49. Thus, the 4031301 protein includes a signal peptide region 1-48 (SEQ ID NO:138) and a polypeptide having the amino acid sequence 49-159 (SEQ ID NO:139), which lacks the signal peptide region.

The amino acid sequence of 4031301 protein was searched against the GenBank database using the BLASTP search protocol. The search identified no significant similarity to any known protein. The predicted encoded protein has a very high probability of localization in the mitochondrial matrix and/or the mitochondrial intermembrane space, and of being secreted through the mitochondrial inner membrane.

Clone 4030250

The nucleic acid provided by clone 4030250 is 590 nucleotides in length and includes an open reading frame encoding a secreted protein having 126 residues (also referred to herein as “4030250protein”) from nucleotides 86 to 463 (SEQ ID NO:140). A signal peptide cleavage site is predicted between residues 66 and 67. Thus, the 4030250 protein includes a signal peptide region 1-66 (SEQ ID NO:141) and a polypeptide having the amino acid sequence 67-126 (SEQ ID NO:142), which lacks the signal peptide region.

The amino acid sequence of 4030250 protein was searched against the GenBank database using the BLASTP search protocol. The search identified a moderate similarity to a portion of the human 5-hydroxytryptamine (serotonin) 5A receptor, a protein of 357 residues. The predicted encoded protein has a high probability of being secreted through the plasma membrane.

Expression of 4030250 nucleic acid sequences is observed in fetal tissues, including brain, liver and kidney, as well as in adult tissues. The adult tissues include liver, adrenal gland and regions of the brain (cerebellum, hippocampus and hypothalamus). Very weak expression of this gene is seen in tumor cell lines

Clone 4160981

The nucleic acid provided by clone 4160981 is 1667 nucleotides in length and includes an open reading frame encoding a secreted protein having 126 residues (also referred to herein as “4160981protein”) from nucleotides 86 to 463 (SEQ ID NO:143). No signal peptide cleavage site was identified in the protein sequence.

The amino acid sequence of 4160981 protein was searched against the GenBank database using the BLASTP search protocol. The search identified a moderate similarity to a fragment from orangutan mitochondrial NADH dehydrogenase (ubiquinone) (EC 1.6.5.3) chain 5. The predicted encoded protein has a high probability of being secreted through the plasma membrane.

Clones 4192452-1 and 4192452-2

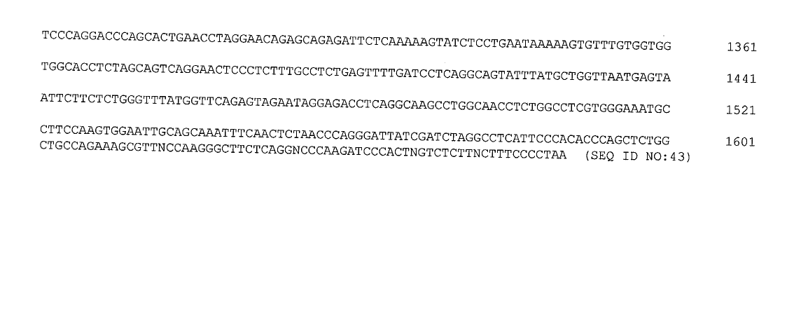

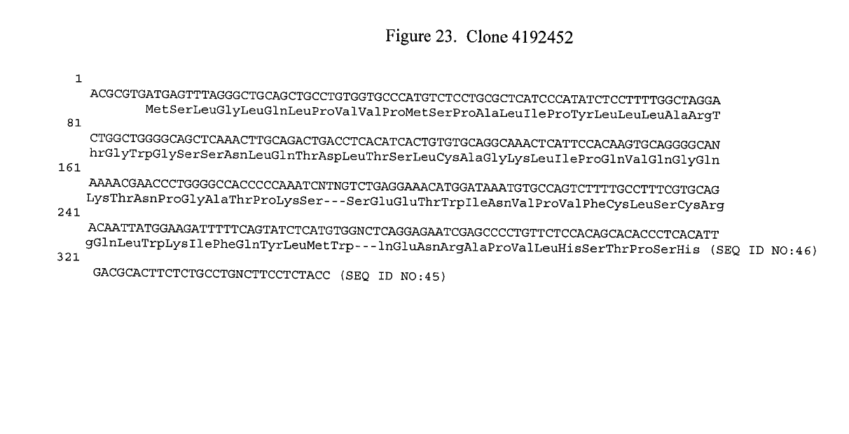

Two nucleic acids differing in the 3′ untranslated region are given clones 4192452-1 and 4192452-2 (FIGS. 23A; SEQ ID NOs: 37, and 155 and 156). These nucleic acids include an open reading frame encoding a secreted protein having 104 residues (also referred to herein as “4192452 protein”) from nucleotides 8 to 319 (SEQ ID NO:144). A signal peptide cleavage site is predicted between residues 28 and 29. Thus, the 4192452 protein includes a signal peptide region having amino acid sequences 1-28 (SEQ ID NO:145) and a polypeptide having the amino acid sequence 29-104 (SEQ ID NO:146), which lacks the signal peptide region.

The amino acid sequence of 4192452 protein was searched against the GenBank database using the BLASTP search protocol. The search identified a weak similarity to a fragment from protein T7N9.6 from Arabidopsis thaliana, a protein of 563 residues. The predicted encoded protein has a moderate probability of being secreted through the plasma membrane.

The present invention discloses SECX nucleic acids, isolated nucleic acids that encode SECX polypeptides or portions thereof, SECX polypeptides, vectors containing these nucleic acids, host cells transformed with the SCX nucleic acids, anti-SECX antibodies, and pharmaceutical compositions. Also disclosed are methods of making SECX polypeptides, as well as methods of screening, diagnosing, treating conditions using these compounds, and methods of screening compounds that modulate SECX polypeptide activity.

SECX Nucleic Acids

One aspect of the invention pertains to isolated nucleic acid molecules that encode SECX proteins or biologically active portions thereof. Also included are nucleic acid fragments sufficient for use as hybridization probes to identify SECX-encoding nucleic acids (e.g., SECX mRNA) and fragments for use as polymerase chain reaction (PCR) primers for the amplification or mutation of SECX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i e., sequences located at the 5′ and 3′ ends of the nucleic acid) in the genomic DNA of the organism from which the nucleic acid is derived. For example, in various embodiments, the isolated SECX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, or a complement of any of these nucleotide sequences, can be isolated using standard molecular biology techniques and the sequence information provided herein. Using all or a portion of these nucleic acid sequences as a hybridization probe, SECX nucleic acid sequences can be isolated using standard hybridization and cloning techniques (e.g., as described in Sambrook et al., eds., Molecular Cloning: a Laboratory Manual 2^{nd} Ed., Cold Spring Harbor Laboratory Press, Cold Spring Harbor, N.Y., 1989; and Ausubel, et al., eds., Current Protocols in Molecular Biology, John Wiley & Sons, New York, N.Y., 1993.)

In some embodiments, the SECX coding sequences include, e.g, the nucleic acid sequence of SEQ ID NOs: 147, 92, 148, 95, 96, 97, 100, 103, 106, 111, 112, 115, 116, 119, 122, 125, 128, 131, 134, 137, 140, 143, or 144, or a complement of any of these sequences.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SECX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in 1, 3, 5, 7, 9, 11, 13, 15, 17, 19, 21, 23, 25, 27, 29, 31, 33, 35, 37, 39, 41, 43, 45, 47, 149, 151, 153, 155, or 156 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, e.g., a fragment that can be used as a probe or primer or a fragment encoding a biologically active portion of SECX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a SECX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a SECX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human SECX protein. Homologous nucleic acid sequences include those nucleic acid sequences that encode conservative amino acid substitutions (see below) in a SECX polypeptide, as well as a polypeptide having a SECX activity. A homologous amino acid sequence does not encode the amino acid sequence of a human SECX polypeptide.

The nucleotide sequence determined from the cloning of human SECX genes allows for the generation of probes and primers designed for use in identifying and/or cloning SECX homologues in other cell types, e.g., from other tissues, as well as SECX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156; or an anti-sense strand nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156; or of a naturally occurring mutant of these sequences.

Probes based on human SECX nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a SECX protein, such as by measuring a level of a SECX-encoding nucleic acid in a sample of cells from a subject e.g., detecting SECX mRNA levels or determining whether a genomic SECX gene has been mutated or deleted.

“A polypeptide having a biologically active portion of SECX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of SECX” can be prepared by isolating a portion of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, that encodes a polypeptide having a SECX biological activity, expressing the encoded portion of SECX protein (e.g., by recombinant expression in vitro) and assessing the activity of the encoded portion of SECX. For example, a nucleic acid fragment encoding a biologically active portion of a SECX polypeptide can optionally include an ATP-binding domain. In another embodiment, a nucleic acid fragment encoding a biologically active portion of SECX includes one or more regions.

SECX Variants

The invention further encompasses nucleic acid molecules that differ from the nucleotide sequences shown in FIGS. 1-23 due to degeneracy of the genetic code. These nucleic acids thus encode the same SECX protein as that encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156. In another embodiment, an isolated nucleic acid molecule of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 150, 152, or 154.

In addition to the human SECX nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, it will be appreciated by those skilled in the art that DNA sequence polymorphisms that lead to changes in the amino acid sequences of a SECX polypeptide may exist within a population (e.g., the human population). Such genetic polymorphism in the SECX gene may exist among individuals within a population due to natural allelic variation. As used herein, the terms “gene” and “recombinant gene” refer to nucleic acid molecules comprising an open reading frame encoding a SECX protein, preferably a mammalian SECX protein. Such natural allelic variations can typically result in 1-5% variance in the nucleotide sequence of the SECX gene. Any and all such nucleotide variations and resulting amino acid polymorphisms in SECX that are the result of natural allelic variation and that do not alter the functional activity of SECX are intended to be within the scope of the invention.

Moreover, nucleic acid molecules encoding SECX proteins from other species, and thus that have a nucleotide sequence that differs from the human sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, are intended to be within the scope of the invention. Nucleic acid molecules corresponding to natural allelic variants and homologues of the SECX DNAs of the invention can be isolated based on their homology to the human SECX nucleic acids disclosed herein using the human cDNAs, or a portion thereof, as a hybridization probe according to standard hybridization techniques under stringent hybridization conditions. For example, a soluble human SECXDNA can be isolated based on its homology to human membrane-bound SECX. Likewise, a membrane-bound human SECXDNA can be isolated based on its homology to soluble human SECX.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding SECX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, Current Protocols in Molecular Biology, John Wiley & Sons, NY, and Kriegler, 1990, Gene Transfer and Expression, a Laboratory Manual, Stockton Press, NY.

In a third embodiment, a nucleic acid that is hybridizable to the nucleic acid molecule comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, Current Transfer and Expression, a Biology, John Wiley & Sons, NY, and Kriegler, 1990, Gene Transfer and Expression, a Laboratory Manual, Stockton Press, NY; Shilo et al., 1981, Proc Natl Acad Sci USA 78: 6789-6792.

Conservative Mutations

In addition to naturally-occurring allelic variants of the SECX sequence that may exist in the population, the skilled artisan will further appreciate that changes can be introduced into a SECX nucleic acid or directly into a SECX polypeptide sequence without altering the functional ability of the SECX protein. In some embodiments, the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 will be altered, thereby leading to changes in the amino acid sequence of the encoded SECX protein. For example, nucleotide substitutions that result in amino acid substitutions at various “non-essential” amino acid residues can be made in the sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156. A “non-essential” amino acid residue is a residue that can be altered from the wild-type sequence of SECX without altering the biological activity, whereas an “essential” amino acid residue is required for biological activity. For example, amino acid residues that are conserved among the SECX proteins of the present invention, are predicted to be particularly unamenable to alteration.

In addition, amino acid residues that are conserved among family members of the SECX proteins of the present invention, are also predicted to be particularly unamenable to alteration. As such, these conserved domains are not likely to be amenable to mutation. Other amino acid residues, however, (e.g., those that are not conserved or only semi-conserved among members of the SECX proteins) may not be essential for activity and thus are likely to be amenable to alteration.

Another aspect of the invention pertains to nucleic acid molecules encoding SECX proteins that contain changes in amino acid residues that are not essential for activity. Such SECX proteins differ in amino acid sequence from SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154, yet retain biological activity. In one embodiment, the isolated nucleic acid molecule comprises a nucleotide sequence encoding a protein, wherein the protein comprises an amino acid sequence at least about 45% homologous to the amino acid sequence of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154. Preferably, the protein encoded by the nucleic acid molecule is at least about 60% homologous to SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154, more preferably at least about 70%, 80%, 90%, 95%, 98%, and most preferably at least about 99% homologous to SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154.

An isolated nucleic acid molecule encoding a SECX protein homologous to the protein of SEQ ID NO:2, 4, 6, 8, 10, 12, 14, 16, 18, 20, 22, 24, 26, 28, 30, 32, 34, 36, 38, 40, 42, 44, 46, 48, 150, 152, or 154 can be created by introducing one or more nucleotide substitutions, additions or deletions into the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, such that one or more amino acid substitutions, additions or deletions are introduced into the encoded protein.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in SECX is replaced with another amino acid residue from the same side chain family. Alternatively, in another embodiment, mutations can be introduced randomly along all or part of a SECX coding sequence, such as by saturation mutagenesis, and the resultant mutants can be screened for SECX biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, the encoded protein can be expressed by any recombinant technology known in the art and the activity of the protein can be determined.

In one embodiment, a mutant SECX protein can be assayed for (1) the ability to form protein:protein interactions with other SECX proteins, other cell-surface proteins, or biologically active portions thereof, (2) complex formation between a mutant SECX protein and a SECX ligand; (3) the ability of a mutant SECX protein to bind to an intracellular target protein or biologically active portion thereof; (e.g., avidin proteins); (4) the ability to bind ATP; or (5) the ability to specifically bind a SECX protein antibody.

Antisense

Another aspect of the invention pertains to isolated antisense nucleic acid molecules that are hybridizable to or complementary to the nucleic acid molecule comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire SECXoding strand, or to only a portion thereof. Nucleic acid molecules encoding fragments, homologs, derivatives and analogs of a SECX protein, e.g., having the amino acid sequences of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154 or antisense nucleic acids complementary to a SECX nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 are additionally provided.

In one embodiment, an antisense nucleic acid molecule is antisense to a “coding region” of the coding strand of a nucleotide sequence encoding SECX. The term “coding region” refers to the region of the nucleotide sequence comprising codons which are translated into amino acid residues (e.g., the protein coding region of Clone 3277789 (SEQ ID NO:9) includes nucleotides 2 to 448). In another embodiment, the antisense nucleic acid molecule is antisense to a “noncoding region” of the coding strand of a nucleotide sequence encoding SECX. The term “noncoding region” refers to 5′ and 3′ sequences which flank the coding region that are not translated into amino acids (i.e., also referred to as 5′ and 3′ untranslated regions).

Given the coding strand sequences encoding SECX disclosed herein (e.g., SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156), antisense nucleic acids of the invention can be designed according to the rules of Watson and Crick or Hoogsteen base pairing. The antisense nucleic acid molecule can be complementary to the entire coding region of SECX mRNA, but more preferably is an oligonucleotide that is antisense to only a portion of the coding or noncoding region of SECX mRNA. For example, the antisense oligonucleotide can be complementary to the region surrounding the translation start site of SECX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SECX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SECX mRNA transcripts to thereby inhibit translation of SECX mRNA. A ribozyme having specificity for a SECX-encoding nucleic acid can be designed based upon the nucleotide sequence of a SECX DNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156). For example, a derivative of a Tetrahymena L-19IVS RNA can be constructed in which the nucleotide sequence of the active site is complementary to the nucleotide sequence to be cleaved in a SECX-encoding mRNA. See, e.g., Cech et al. U.S. Pat. No. 4,987,071; and Cech et al. U.S. Pat. No. 5,116,742. Alternatively, SECX mRNA can be used to select a catalytic RNA having a specific ribonuclease activity from a pool of RNA molecules. See, e.g., Bartel et al., (1993) Science 261:1411-1418.

Alternatively, SECX gene expression can be inhibited by targeting nucleotide sequences complementary to the regulatory region of a SECX nucleic acid (e.g., the SECX promoter and/or enhancers) to form triple helical structures that prevent transcription of the SECX gene in target cells. See generally, Helene. (1991) Anticancer Drug Des. 6: 569-84; Helene. et al. (1992) Ann. N.Y Acad. Sci 660:27-36; and Maher (1992) Bioassays 14: 807-15.

In various embodiments, the nucleic acids of SECX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of SECX can be used in therapeutic and diagnostic applications. For example, PNAs can be used as antisense or antigene agents for sequence-specific modulation of gene expression by, e.g., inducing transcription or translation arrest or inhibiting replication. PNAs of SECX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of SECX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of SECX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

SECX Polypeptides

One aspect of the invention pertains to isolated SECX proteins, and biologically active portions thereof, or derivatives, fragments, analogs or homologs thereof. Also provided are polypeptide fragments suitable for use as immunogens to raise anti-SECX antibodies. In one embodiment, native SECX proteins can be isolated from cells or tissue sources by an appropriate purification scheme using standard protein purification techniques. In another embodiment, SECX proteins are produced by recombinant DNA techniques. Alternative to recombinant expression, a SECX protein or polypeptide can be synthesized chemically using standard peptide synthesis techniques.

An “isolated” or “purified” protein or biologically active portion thereof is substantially free of cellular material or other contaminating proteins from the cell or tissue source from which the SECX protein is derived, or substantially free from chemical precursors or other chemicals when chemically synthesized. The language “substantially free of cellular material” includes preparations of SECX protein in which the protein is separated from cellular components of the cells from which it is isolated or recombinantly produced. In one embodiment, the language “substantially free of cellular material” includes preparations of SECX protein having less than about 30% (by dry weight) of non-SECX protein (also referred to herein as a “contaminating protein”), more preferably less than about 20% of non-SECX protein, still more preferably less than about 10% of non-SECX protein, and most preferably less than about 5% non-SECX protein. When the SECX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SECX protein in which the protein is separated from chemical precursors or other chemicals that are involved in the synthesis of the protein. In one embodiment, the language “substantially free of chemical precursors or other chemicals” includes preparations of SECX protein having less than about 30% (by dry weight) of chemical precursors or non-SECX chemicals, more preferably less than about 20% chemical precursors or non-SECX chemicals, still more preferably less than about 10% chemical precursors or non-SECX chemicals, and most preferably less than about 5% chemical precursors or non-SECX chemicals.

Biologically active portions of a SECX protein include peptides comprising amino acid sequences sufficiently homologous to or derived from the amino acid sequence of the SECX protein, e.g., the amino acid sequence shown in SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154 that include fewer amino acids than the full length SECX proteins, and exhibit at least one activity of a SECX protein. Typically, biologically active portions comprise a domain or motif with at least one activity of the SECX protein. A biologically active portion of a SECX protein can be a polypeptide which is, for example, 10, 25, 50, 100 or more amino acids in length.

A biologically active portion of a SECX protein of the present invention may contain at least one of the above-identified domains conserved between the SECX proteins. An alternative biologically active portion of a SECX protein may contain at least two of the above-identified domains. Another biologically active portion of a SECX protein may contain at least three of the above-identified domains. Yet another biologically active portion of a SECX protein of the present invention may contain at least four of the above-identified domains.

Moreover, other biologically active portions, in which other regions of the protein are deleted, can be prepared by recombinant techniques and evaluated for one or more of the functional activities of a native SECX protein.

In an embodiment, the SECX protein has an amino acid sequence shown in SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154. In other embodiments, the SECX protein is substantially homologous to one of these SECX proteins and retains its the functional activity, yet differs in amino acid sequence due to natural allelic variation or mutagenesis, as described in detail below. Accordingly, in another embodiment, the SECX protein is a protein that comprises an amino acid sequence at least about 45% homologous to the amino acid sequence of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154 and retains the functional activity of the SECX protein.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides SECX chimeric or fusion proteins. As used herein, a SECX “chimeric protein” or “fusion protein” comprises a SECX polypeptide operatively linked to a non-SECX polypeptide. A “SECX polypeptide” refers to a polypeptide having an amino acid sequence corresponding to SECX, whereas a “non-SECX polypeptide” refers to a polypeptide having an amino acid sequence corresponding to a protein that is not substantially homologous to the SECX protein, e.g., a protein that is different from the SECX protein and that is derived from the same or a different organism. Within a SECX fusion protein the SECX polypeptide can correspond to all or a portion of a SECX protein. In one embodiment, a SECX fusion protein comprises at least one biologically active portion of a SECX protein. In another embodiment, a SECX fusion protein comprises at least two biologically active portions of a SECX protein. In yet another embodiment, a SECX fusion protein comprises at least three biologically active portions of a SECX protein. Within the fusion protein, the term “operatively linked” is intended to indicate that the SECX polypeptide and the non-SECX polypeptide are fused in-frame to each other. The non-SECX polypeptide can be fused to the N-terminus or C-terminus of the SECX polypeptide.

For example, in one embodiment a SECX fusion protein comprises a SECX domain operably linked to the extracellular domain of a second protein. Such fusion proteins can be further utilized in screening assays for compounds which modulate SECX activity (such assays are described in detail below).

In yet another embodiment, the fusion protein is a GST-SECX fusion protein in which the SECX sequences are fused to the C-terminus of the GST (i.e., glutathione S-transferase) sequences. Such fusion proteins can facilitate the purification of recombinant SECX.

In another embodiment, the fusion protein is a SECX protein containing a heterologous signal sequence at its N-terminus. For example, a native SECX signal sequence can be removed and replaced with a signal sequence from another protein. In certain host cells (e.g., mammalian host cells), expression and/or secretion of SECX can be increased through use of a heterologous signal sequence.

In yet another embodiment, the fusion protein is a SECX-immunoglobulin fusion protein in which the SECX sequences comprising one or more domains are fused to sequences derived from a member of the immunoglobulin protein family. The SECX-immunoglobulin fusion proteins of the invention can be incorporated into pharmaceutical compositions and administered to a subject to inhibit an interaction between a SECX ligand and a SECX protein on the surface of a cell, to thereby suppress SECX-mediated signal transduction in vivo. The SECX-immunoglobulin fusion proteins can be used to affect the bioavailability of a SECX cognate ligand. Inhibition of the SECX ligand/SECX interaction may be useful therapeutically for both the treatment of proliferative and differentiative disorders, as well as modulating (e.g. promoting or inhibiting) cell survival. Moreover, the SECX-immunoglobulin fusion proteins of the invention can be used as immunogens to produce anti-SECX antibodies in a subject, to purify SECX ligands, and in screening assays to identify molecules that inhibit the interaction of SECX with a SECX ligand.

A SECX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that already encode a fusion moiety (e.g., a GST polypeptide). A SECX-encoding nucleic acid can be cloned into such an expression vector such that the fusion moiety is linked in-frame to the SECX protein.

The invention also provides signal sequences derived from various SECX polypeptides. The signal sequences include, e.g., polypeptides including a portion of SEQ ID NOs:90, 93, 98, 101, 104, 107, 109, 113, 117, 120, 123, 126, 129, 132, 135, 138, 141, and 145. In some embodiments, the signal sequence includes a portion of a SECX signal sequence that is sufficient to direct a linked polypeptide to a desired cellular compartment.

SECX Agonists and Antagonists

The present invention also pertains to variants of the SECX proteins that function as either SECX agonists (mimetics) or as SECX antagonists. Variants of the SECX protein can be generated by mutagenesis, e.g., discrete point mutation or truncation of the SECX protein. An agonist of the SECX protein can retain substantially the same, or a subset of, the biological activities of the naturally occurring form of the SECX protein. An antagonist of the SECX protein can inhibit one or more of the activities of the naturally occurring form of the SECX protein by, for example, competitively binding to a downstream or upstream member of a cellular signaling cascade which includes the SECX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the SECX proteins.

Variants of the SECX protein that function as either SECX agonists (mimetics) or as SECX antagonists can be identified by screening combinatorial libraries of mutants, e.g., truncation mutants, of the SECX protein for SECX protein agonist or antagonist activity. In one embodiment, a variegated library of SECX variants is generated by combinatorial mutagenesis at the nucleic acid level and is encoded by a variegated gene library. A variegated library of SECX variants can be produced by, for example, enzymatically ligating a mixture of synthetic oligonucleotides into gene sequences such that a degenerate set of potential SECX sequences is expressible as individual polypeptides, or alternatively, as a set of larger fusion proteins (e.g., for phage display) containing the set of SECX sequences therein. There are a variety of methods which can be used to produce libraries of potential SECX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SECX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the SECX protein coding sequence can be used to generate a variegated population of SECX fragments for screening and subsequent selection of variants of a SECX protein. In one embodiment, a library of coding sequence fragments can be generated by treating a double stranded PCR fragment of a SECX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the SECX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SECX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify SECX variants (Arkin and Yourvan (1992) PNAS 89:7811-7815; Delgrave et al. (1993) Protein Engineering 6:327-331).

Anti-SECX Antibodies

An isolated SECX protein, or a portion or fragment thereof, can be used as an immunogen to generate antibodies that bind SECX using standard techniques for polyclonal and monoclonal antibody preparation. The full-length SECX protein can be used or, alternatively, the invention provides antigenic peptide fragments of SECX for use as immunogens. The antigenic peptide of SECXomprises at least 8 amino acid residues of the amino acid sequence shown in SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154 and encompasses an epitope of SECX such that an antibody raised against the peptide forms a specific immune complex with SECX. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of SECX that are located on the surface of the protein, e.g., hydrophilic regions.

As disclosed herein, SECX protein sequence of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154, or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, F_{ab} and F_{(ab′)2} fragments, and an F_{ab} expression library. Various procedures known within the art may be used for the production of polyclonal or monoclonal antibodies to a SECX protein sequence of SEQ ID NOs: 2, 4, 6, 8, 10, 12, 14, 16, 18, 20, 22, 24, 26, 28, 30, 32, 34, 36, 38, 40, 42, 44, 46, 48, 150, 152, or 154, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed SECX protein or a chemically synthesized SECX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against SECXan be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of SECX. A monoclonal antibody composition thus typically displays a single binding affinity for a particular SECX protein with which it immunoreacts. For preparation of monoclonal antibodies directed towards a particular SECX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al, 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

According to the invention, techniques can be adapted for the production of single-chain antibodies specific to a SECX protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a SECX protein or derivatives, fragments, analogs or homologs thereof. Non-human antibodies can be “humanized” by techniques well known in the art. See e.g., U.S. Pat. No. 5,225,539. Antibody fragments that contain the idiotypes to a SECX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-SECX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst. 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a SECX protein is facilitated by generation of hybridomas that bind to the fragment of a SECX protein possessing such a domain. Antibodies that are specific for one or more domains within a SECX protein, e.g., domains spanning the above-identified conserved regions of SECX family proteins, or derivatives, fragments, analogs or homologs thereof, are also provided herein.

Anti-SECX antibodies may be used in methods known within the art relating to the localization and/or quantitation of a SECX protein (e.g., for use in measuring levels of the SECX protein within appropriate physiological samples, for use in diagnostic methods, for use in imaging the protein, and the like). In a given embodiment, antibodies for SECX proteins, or derivatives, fragments, analogs or homologs thereof, that contain the antibody derived binding domain, are utilized as pharmacologically-active compounds [hereinafter “Therapeutics”].

An anti-SECX antibody (e.g., monoclonal antibody) can be used to isolate SECX by standard techniques, such as affinity chromatography or immunoprecipitation. An anti-SECX antibody can facilitate the purification of natural SECX from cells and of recombinantly produced SECX expressed in host cells. Moreover, an anti-SECX antibody can be used to detect SECX protein (e.g., in a cellular lysate or cell supernatant) in order to evaluate the abundance and pattern of expression of the SECX protein. Anti-SECX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

SECX Recombinant Expression Vectors and Host Cells

Another aspect of the invention pertains to vectors, preferably expression vectors, containing a nucleic acid encoding SECX protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SECX proteins, mutant forms of SECX, fusion proteins, etc.).

The recombinant expression vectors of the invention can be designed for expression of SECX in prokaryotic or eukaryotic cells. For example, SECX can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the SECX expression vector is a yeast expression vector. Examples of vectors for expression in yeast S. cerevisiae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (In Vitrogen Corp, San Diego, Calif.).

Alternatively, SECX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., Molecular Cloning: a Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to SECX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SECX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: a Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding SECX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an SECX protein. Accordingly, the invention further provides methods for producing SECX protein using the host cells of the invention. In one embodiment, the method comprises culturing the host cell of invention (into which a recombinant expression vector encoding SECX has been introduced) in a suitable medium such that SECX protein is produced. In another embodiment, the method further comprises isolating SECX from the medium or the host cell.

Transgenic Animals

The host cells of the invention can also be used to produce nonhuman transgenic animals. For example, in one embodiment, a host cell of the invention is a fertilized oocyte or an embryonic stem cell into which SECX-coding sequences have been introduced. Such host cells can then be used to create non-human transgenic animals in which exogenous SECX sequences have been introduced into their genome or homologous recombinant animals in which endogenous SECX sequences have been altered. Such animals are useful for studying the function and/or activity of SECX and for identifying and/or evaluating modulators of SECX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SECX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SECX-encoding nucleic acid into the male pronuclei of a fertilized oocyte, e.g., by microinjection, retroviral infection, and allowing the oocyte to develop in a pseudopregnant female foster animal. The human SECX DNA sequence of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 can be introduced as a transgene into the genome of a non-human animal. Alternatively, a nonhuman homologue of the human SECX gene, such as a mouse SECX gene, can be isolated based on hybridization to the human SECXDNA (described further above) and used as a transgene. Intronic sequences and polyadenylation signals can also be included in the transgene to increase the efficiency of expression of the transgene. A tissue-specific regulatory sequence(s) can be operably linked to the SECX transgene to direct expression of SECX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the SECX transgene in its genome and/or expression of SECX mRNA in tissues or cells of the animals. A transgenic founder animal can then be used to breed additional animals carrying the transgene. Moreover, transgenic animals carrying a transgene encoding SECXan further be bred to other transgenic animals carrying other transgenes.

To create a homologous recombinant animal, a vector is prepared which contains at least a portion of a SECX gene into which a deletion, addition or substitution has been introduced to thereby alter, e.g., functionally disrupt, the SECX gene. The SECX gene can be a human gene (e.g., SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156), but more preferably, is a non-human homologue of a human SECX gene. For example, a mouse homologue of human SECX gene of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 can be used to construct a homologous recombination vector suitable for altering an endogenous SECX gene in the mouse genome. In one embodiment, the vector is designed such that, upon homologous recombination, the endogenous SECX gene is functionally disrupted (i.e., no longer encodes a functional protein; also referred to as a “knock out” vector).

Alternatively, the vector can be designed such that, upon homologous recombination, the endogenous SECX gene is mutated or otherwise altered but still encodes functional protein (e.g., the upstream regulatory region can be altered to thereby alter the expression of the endogenous SECX protein). In the homologous recombination vector, the altered portion of the SECX gene is flanked at its 5′ and 3′ ends by additional nucleic acid of the SECX gene to allow for homologous recombination to occur between the exogenous SECX gene carried by the vector and an endogenous SECX gene in an embryonic stem cell. The additional flanking SECX nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced SECX gene has homologously recombined with the endogenous SECX gene are selected (see e.g., Li et al. (1992) Cell 69:915).

The selected cells are then injected into a blastocyst of an animal (e.g., a mouse) to form aggregation chimeras. See e.g., Bradley 1987, In: Teratocarcinomas and Embryonic Stem Cells: a Practical Approach, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The SECX nucleic acid molecules, SECX proteins, and anti-SECX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SECX protein or anti-SECX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The nucleic acid molecules of the invention can be inserted into vectors and used as gene therapy vectors. Gene therapy vectors can be delivered to a subject by any of a number of routes, e.g., as described in U.S. Pat. No. 5,703,055. Delivery can thus also include, e.g., intravenous injection, local administration (see U.S. Pat. No. 5,328,470) or stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology), (c) predictive medicine (e.g., diagnostic assays, prognostic assays, monitoring clinical trials, and pharmacogenomics); and (d) methods of treatment (e.g., therapeutic and prophylactic).

The detection assays can be based on tissues in which altered levels of expression of a SECX nucleic acid are detected. For example, SECX gene 2826468 shows expression in the cerebllum and hippocampus regions of brain. Accordingly, high levels of expression of this sequence can indicate the presence of this tissue. Gene 2826468 is also observed at high levels in colon cancer cell lines, and can thus be used to determine if a sample tissue is cancerous. Similarly, gene 3186754 is expressed at high levels in heart, skeletal muscle, brain and spinal cord. The presence of transcribed 3186754 sequences can indicate the presence of these tissues. Conversely, 3186754 sequences are underrepresented in tumor cell lines. Therefore, the absence of this transcript in a tissue sample can indicate that the tumor cell is cancerous. Other SECX genes, such as 3277237, 3487483, 3540920, 3903091, and 4030250, similarly show altered expression levels in various tissue types, as discussed below. Altered levels of one or more of these SECX sequences can be used to identify a particular tissue.

The isolated nucleic acid molecules of the invention can be used to express SECX protein (e.g., via a recombinant expression vector in a host cell in gene therapy applications), to detect SECX mRNA (e.g., in a biological sample) or a genetic lesion in a SECX gene, and to modulate SECX activity, as described further below. In addition, the SECX proteins can be used to screen drugs or compounds that modulate the SECX activity or expression as well as to treat disorders characterized by insufficient or excessive production of SECX protein, e.g., cancers or neurological conditions, or production of SECX protein forms that have decreased or aberrant activity compared to SECX wild type protein. In addition, the anti-SECX antibodies of the invention can be used to detect and isolate SECX proteins and modulate SECX activity.

This invention further pertains to novel agents identified by the above described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) that bind to SECX proteins or have a stimulatory or inhibitory effect on, for example, SECX expression or SECX activity.

In one embodiment, the invention provides assays for screening candidate or test compounds which bind to or modulate the activity of a SECX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994)J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of SECX protein, or a biologically active portion thereof, on the cell surface is contacted with a test compound and the ability of the test compound to bind to a SECX protein determined. The cell, for example, can of mammalian origin or a yeast cell. Determining the ability of the test compound to bind to the SECX protein can be accomplished, for example, by coupling the test compound with a radioisotope or enzymatic label such that binding of the test compound to the SECX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of SECX protein, or a biologically active portion thereof, on the cell surface with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or a biologically active portion thereof as compared to the known compound.

In another embodiment, an assay is a cell-based assay comprising contacting a cell expressing a membrane-bound form of SECX protein, or a biologically active portion thereof, on the cell surface with a test compound and determining the ability of the test compound to modulate (e.g., stimulate or inhibit) the activity of the SECX protein or biologically active portion thereof. Determining the ability of the test compound to modulate the activity of SECX or a biologically active portion thereof can be accomplished, for example, by determining the ability of the SECX protein to bind to or interact with a SECX target molecule. As used herein, a “target molecule” is a molecule with which a SECX protein binds or interacts in nature, for example, a molecule on the surface of a cell which expresses a SECX protein, a molecule on the surface of a second cell, a molecule in the extracellular milieu, a molecule associated with the internal surface of a cell membrane or a cytoplasmic molecule. A SECX target molecule can be a non-SECX molecule or a SECX protein or polypeptide of the present invention. In one embodiment, a SECX target molecule is a component of a signal transduction pathway that facilitates transduction of an extracellular signal (e.g., a signal generated by binding of a compound to a membrane-bound SECX molecule) through the cell membrane and into the cell. The target, for example, can be a second intercellular protein that has catalytic activity or a protein that facilitates the association of downstream signaling molecules with SECX.

Determining the ability of the SECX protein to bind to or interact with a SECX target molecule can be accomplished by one of the methods described above for determining direct binding. In one embodiment, determining the ability of the SECX protein to bind to or interact with a SECX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a SECX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a SECX protein or biologically active portion thereof with a test compound and determining the ability of the test compound to bind to the SECX protein or biologically active portion thereof. Binding of the test compound to the SECX protein can be determined either directly or indirectly as described above. In one embodiment, the assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or biologically active portion thereof as compared to the known compound.

In another embodiment, an assay is a cell-free assay comprising contacting SECX protein or biologically active portion thereof with a test compound and determining the ability of the test compound to modulate (e.g., stimulate or inhibit) the activity of the SECX protein or biologically active portion thereof. Determining the ability of the test compound to modulate the activity of SECXan be accomplished, for example, by determining the ability of the SECX protein to bind to a SECX target molecule by one of the methods described above for determining direct binding. In an alternative embodiment, determining the ability of the test compound to modulate the activity of SECXan be accomplished by determining the ability of the SECX protein further modulate a SECX target molecule. For example, the catalytic/enzymatic activity of the target molecule on an appropriate substrate can be determined as previously described.

In yet another embodiment, the cell-free assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the SECX protein to preferentially bind to or modulate the activity of a SECX target molecule.

The cell-free assays of the present invention are amenable to use of both the soluble form or the membrane-bound form of SECX. In the case of cell-free assays comprising the membrane-bound form of SECX, it may be desirable to utilize a solubilizing agent such that the membrane-bound form of SECX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-(3-cholamidopropyl)dimethylamminiol-1-propane sulfonate (CHAPS), 3-(3-cholamidopropyl)dimethylamminiol-2-hydroxy-1-propane sulfonate (CHAPSO), or N-dodecyl-N,N-dimethyl-3-ammonio-1-propane sulfonate.

In more than one embodiment of the above assay methods of the present invention, it may be desirable to immobilize either SECX or its target molecule to facilitate separation of complexed from uncomplexed forms of one or both of the proteins, as well as to accommodate automation of the assay. Binding of a test compound to SECX, or interaction of SECX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-SECX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or SECX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SECX binding or activity determined using standard techniques.

Other techniques for immobilizing proteins on matrices can also be used in the screening assays of the invention. For example, either SECX or its target molecule can be immobilized utilizing conjugation of biotin and streptavidin. Biotinylated SECX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SECX or target molecules, but which do not interfere with binding of the SECX protein to its target molecule, can be derivatized to the wells of the plate, and unbound target or SECX trapped in the wells by antibody conjugation. Methods for detecting such complexes, in addition to those described above for the GST-immobilized complexes, include immunodetection of complexes using antibodies reactive with the SECX or target molecule, as well as enzyme-linked assays that rely on detecting an enzymatic activity associated with the SECX or target molecule.

In another embodiment, modulators of SECX expression are identified in a method wherein a cell is contacted with a candidate compound and the expression of SECX mRNA or protein in the cell is determined. The level of expression of SECX mRNA or protein in the presence of the candidate compound is compared to the level of expression of SECX mRNA or protein in the absence of the candidate compound. The candidate compound can then be identified as a modulator of SECX expression based on this comparison. For example, when expression of SECX mRNA or protein is greater (statistically significantly greater) in the presence of the candidate compound than in its absence, the candidate compound is identified as a stimulator of SECX mRNA or protein expression. Alternatively, when expression of SECX mRNA or protein is less (statistically significantly less) in the presence of the candidate compound than in its absence, the candidate compound is identified as an inhibitor of SECX mRNA or protein expression. The level of SECX mRNA or protein expression in the cells can be determined by methods described herein for detecting SECX mRNA or protein.

In yet another aspect of the invention, the SECX proteins can be used as “bait proteins” in a two-hybrid assay or three hybrid assay (see, e.g., U.S. Pat. No. 5,283,317; Zervos et al. (1 993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with SECX(“SECX-binding proteins” or “SECX-bp”) and modulate SECX activity. Such SECX-binding proteins are also likely to be involved in the propagation of signals by the SECX proteins as, for example, upstream or downstream elements of the SECX pathway.

The two-hybrid system is based on the modular nature of most transcription factors, which consist of separable DNA-binding and activation domains. Briefly, the assay utilizes two different DNA constructs. In one construct, the gene that codes for SECX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SECX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with SECX.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

Chromosome Mapping

Once the sequence (or a portion of the sequence) of a gene has been isolated, this sequence can be used to map the location of the gene on a chromosome. This process is called chromosome mapping. Accordingly, portions or fragments of the SECX, sequences, described herein, can be used to map the location of the SECX genes, respectively, on a chromosome. The mapping of the SECX sequences to chromosomes is an important first step in correlating these sequences with genes associated with disease.

Briefly, SECX genes can be mapped to chromosomes by preparing PCR primers (preferably 15-25 bp in length) from the SECX sequences. Computer analysis of the SECX, sequences can be used to rapidly select primers that do not span more than one exon in the genomic DNA, thus complicating the amplification process. These primers can then be used for PCR screening of somatic cell hybrids containing individual chromosomes of a given species. Only those hybrids containing the species-specific gene corresponding to the SECX sequences will yield an amplified fragment.

PCR mapping of somatic cell hybrids is a rapid procedure for assigning a particular sequence to a particular chromosome. Three or more sequences can be assigned per day using a single thermal cycler. Using the SECX sequences to design oligonucleotide primers, sublocalization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see Verma et al., Human Chromosomes: a Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, for example, in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the SECX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The SECX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the SECX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The SECX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156 can provide positive individual identification with a panel of perhaps 10 to 1,000 primers that each yield a noncoding amplified sequence of 100 bases. If predicted coding sequences are used, a more appropriate number of primers for positive individual identification would be 500-2,000.

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression or activity.

Another aspect of the invention provides methods for determining SECX protein, nucleic acid expression or SECX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of SECX in clinical trials.

These and other agents are described in further detail in the following sections.

Diagnostic Assays

An exemplary method for detecting the presence or absence of SECX in a biological sample involves obtaining a biological sample from a test subject and contacting the biological sample with a compound or an agent capable of detecting SECX protein or nucleic acid (e.g., mRNA, genomic DNA) that encodes SECX protein such that the presence of SECX is detected in the biological sample. An agent for detecting SECX mRNA or genomic DNA is a labeled nucleic acid probe capable of hybridizing to SECX mRNA or genomic DNA. The nucleic acid probe can be, for example, a full-length SECX nucleic acid, such as the nucleic acid of SEQ ID NO:1, 3, 5, 7, 9, 11, 13, 15, 17, 19, 21, 23, 25, 27, 29, 31, 33, 35, 37, 39, 41, 43, 45, 47, 149, 151, 153, 155, or 156, or a portion thereof, such as an oligonucleotide of at least 15, 30, 50, 100, 250 or 500 nucleotides in length and sufficient to specifically hybridize under stringent conditions to SECX mRNA or genomic DNA. Other suitable probes for use in the diagnostic assays of the invention are described herein.

An agent for detecting SECX protein is an antibody capable of binding to SECX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SECX mRNA, protein, or genomic DNA in a biological sample in vitro as well as in vivo. For example, in vitro techniques for detection of SECX mRNA include Northern hybridizations and in situ hybridizations. In vitro techniques for detection of SECX protein include enzyme linked immunosorbent assays (ELISAs), Western blots, immunoprecipitations and immunofluorescence. In vitro techniques for detection of SECX genomic DNA include Southern hybridizations. Furthermore, in vivo techniques for detection of SECX protein include introducing into a subject a labeled anti-SECX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting SECX protein, mRNA, or genomic DNA, such that the presence of SECX protein, mRNA or genomic DNA is detected in the biological sample, and comparing the presence of SECX protein, mRNA or genomic DNA in the control sample with the presence of SECX protein, mRNA or genomic DNA in the test sample.

The invention also encompasses kits for detecting the presence of SECX in a biological sample. For example, the kit can comprise: a labeled compound or agent capable of detecting SECX protein or mRNA in a biological sample; means for determining the amount of SECX in the sample; and means for comparing the amount of SECX in the sample with a standard. The compound or agent can be packaged in a suitable container. The kit can further comprise instructions for using the kit to detect SECX protein or nucleic acid.

For detecting SECX nucleic acids, the kit can include nucleic acids which hybridize to SECX nucleic acids or which specifically amplify SECX nucleic acids.

Prognostic Assays

The diagnostic methods described herein can furthermore be utilized to identify subjects having or at risk of developing a disease or disorder associated with aberrant SECX expression or activity. For example, the assays described herein, such as the preceding diagnostic assays or the following assays, can be utilized to identify a subject having or at risk of developing a disorder associated with SECX protein, nucleic acid expression or activity in, e.g., cancer, or neurological conditions. Alternatively, the prognostic assays can be utilized to identify a subject having or at risk for developing a disease or disorder. Thus, the present invention provides a method for identifying a disease or disorder associated with aberrant SECX expression or activity in which a test sample is obtained from a subject and SECX protein or nucleic acid (e.g., mRNA, genomic DNA) is detected, wherein the presence of SECX protein or nucleic acid is diagnostic for a subject having or at risk of developing a disease or disorder associated with aberrant SECX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SECX expression or activity. For example, such methods can be used to determine whether a subject can be effectively treated with an agent for a pathological disorder, such as a neurological disorder or cancer. Thus, the present invention provides methods for determining whether a subject can be effectively treated with an agent for a disorder associated with aberrant SECX expression or activity in which a test sample is obtained and SECX protein or nucleic acid is detected (e.g., wherein the presence of SECX protein or nucleic acid is diagnostic for a subject that can be administered the agent to treat a disorder associated with aberrant SECX expression or activity.)

The methods of the invention can also be used to detect genetic lesions in a SECX gene, thereby determining if a subject with the lesioned gene is at risk for, or suffers from, a pathological condition. In various embodiments, the methods include detecting, in a sample of cells from the subject, the presence or absence of a genetic lesion characterized by at least one of an alteration affecting the integrity of a gene encoding a SECX-protein, or the mis-expression of the SECX gene. For example, such genetic lesions can be detected by ascertaining the existence of at least one of (1) a deletion of one or more nucleotides from a SECX gene; (2) an addition of one or more nucleotides to a SECX gene; (3) a substitution of one or more nucleotides of a SECX gene, (4) a chromosomal rearrangement of a SECX gene; (5) an alteration in the level of a messenger RNA transcript of a SECX gene, (6) aberrant modification of a SECX gene, such as of the methylation pattern of the genomic DNA, (7) the presence of a non-wild type splicing pattern of a messenger RNA transcript of a SECX gene, (8) a non-wild type level of a SECX-protein, (9) allelic loss of a SECX gene, and (10) inappropriate post-translational modification of a SECX-protein. As described herein, there are a large number of assay techniques known in the art which can be used for detecting lesions in a SECX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the SECX-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a SECX gene under conditions such that hybridization and amplification of the SECX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SECX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,53 1) can be used to score for the presence of specific mutations by development or loss of a ribozyme cleavage site.

In other embodiments, genetic mutations in SECX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in SECX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a SECX gene and detect mutations by comparing the sequence of a sample SECX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the SECX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type SECX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SECXDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a SECX sequence, e.g., a wild-type SECX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in SECX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control SECX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6: 1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SECX gene.

Furthermore, any cell type or tissue, preferably peripheral blood leukocytes, in which SECX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on an activity of a SECX nucleic acid or protein (e.g., SECX gene expression), as identified by a screening assay described herein can be administered to individuals to treat (prophylactically or therapeutically) disorders (e.g., neurological, cancer-related or gestational disorders) associated with aberrant SECX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of SECX protein, expression of SECX nucleic acid, or mutation content of SECX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of SECX protein, expression of SECX nucleic acid, or mutation content of SECX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a SECX modulator, such as a modulator identified by one of the exemplary screening assays described herein.

Monitoring Clinical Efficacy

Monitoring the influence of agents (e.g., drugs, compounds) on the expression or activity of SECX (e.g., the ability to modulate aberrant cell proliferation and/or differentiation) can be applied not only in basic drug screening, but also in clinical trials. For example, the effectiveness of an agent determined by a screening assay as described herein to increase SECX gene expression, protein levels, or upregulate SECX activity, can be monitored in clinical trials of subjects exhibiting decreased SECX gene expression, protein levels, or downregulated SECX activity. Alternatively, the effectiveness of an agent determined by a screening assay to decrease SECX gene expression, protein levels, or downregulate SECX activity, can be monitored in clinical trials of subjects exhibiting increased SECX gene expression, protein levels, or upregulated SECX activity. In such clinical trials, the expression or activity of SECX and, preferably, other genes that have been implicated in, for example, a neurological disorder, can be used as a “read out” or markers of the immune responsiveness of a particular cell.

For example, genes, including SECX genes, that are modulated in cells by treatment with an agent (e.g., compound, drug or small molecule) that modulates SECX activity (e.g., identified in a screening assay as described herein) can be identified. Thus, to study the effect of agents on cellular proliferation disorders, for example, in a clinical trial, cells can be isolated and RNA prepared and analyzed for the levels of expression of SECX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SECX or other genes. In this way, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a SECX protein, mRNA, or genomic DNA in the preadministration sample; (iii) obtaining one or more post-administration samples from the subject; (iv) detecting the level of expression or activity of the SECX protein, mRNA, or genomic DNA in the post-administration samples; (v) comparing the level of expression or activity of the SECX protein, mRNA, or genomic DNA in the pre-administration sample with the SECX protein, mRNA, or genomic DNA in the post administration sample or samples; and (vi) altering the administration of the agent to the subject accordingly. For example, increased administration of the agent may be desirable to increase the expression or activity of SECX to higher levels than detected, i.e., to increase the effectiveness of the agent. Alternatively, decreased administration of the agent may be desirable to decrease expression or activity of SECX to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant SECX expression or activity.

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) a SECX polypeptide, or analogs, derivatives, fragments or homologs thereof; (ii) antibodies to a SECX peptide; (iii) nucleic acids encoding a SECX peptide; (iv) administration of antisense nucleic acid and nucleic acids that are “dysfunctional” (i.e., due to a heterologous insertion within the coding sequences of coding sequences to a SECX peptide) are utilized to “knockout” endogenous function of a SECX peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between a SECX peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, a SECX peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a SECX peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

In one aspect, the invention provides a method for preventing, in a subject, a disease or condition associated with an aberrant SECX expression or activity, by administering to the subject an agent that modulates SECX expression or at least one SECX activity. Subjects at risk for a disease that is caused or contributed to by aberrant SECX expression or activity can be identified by, for example, any or a combination of diagnostic or prognostic assays as described herein. Administration of a prophylactic agent can occur prior to the manifestation of symptoms characteristic of the SECX aberrancy, such that a disease or disorder is prevented or, alternatively, delayed in its progression. Depending on the type of SECX aberrancy, for example, a SECX agonist or SECX antagonist agent can be used for treating the subject. The appropriate agent can be determined based on screening assays described herein.

Another aspect of the invention pertains to methods of modulating SECX expression or activity for therapeutic purposes. The modulatory method of the invention involves contacting a cell with an agent that modulates one or more of the activities of SECX protein activity associated with the cell. An agent that modulates SECX protein activity can be an agent as described herein, such as a nucleic acid or a protein, a naturally-occurring cognate ligand of a SECX protein, a peptide, a SECX peptidomimetic, or other small molecule. In one embodiment, the agent stimulates one or more SECX protein activity. Examples of such stimulatory agents include active SECX protein and a nucleic acid molecule encoding SECX that has been introduced into the cell. In another embodiment, the agent inhibits one or more SECX protein activity. Examples of such inhibitory agents include antisense SECX nucleic acid molecules and anti-SECX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a SECX protein or nucleic acid molecule. In one embodiment, the method involves administering an agent (e.g., an agent identified by a screening assay described herein), or combination of agents that modulates (e.g., upregulates or downregulates) SECX expression or activity. In another embodiment, the method involves administering a SECX protein or nucleic acid molecule as therapy to compensate for reduced or aberrant SECX expression or activity.

### EXAMPLES

The invention will be further described in the following examples, which do not limit the scope of the invention described in the claims. The following examples illustrate the characterization of SECX nucleic acids and polypeptides.

### Example 1

Construction of a Vector for Expressing SECX Nucleic Acid Sequences

An expression vector, named pBIgHis, was constructed for expressing SECX nucleic acid sequences. To construct the pBIgHis expression vector, oligonucleotide primers were designed to amplify the Fc fragment of the human immunoglobulin heavy chain. The forward primer was 5′-CCGCTCGAGTGAGCCCAAATCTTGTGACAAA (SEQ ID NO:47), and the reverse primer was 5′-GCTCTAGACTTTTACCCGGGGACAGGGAG (SEQ ID NO:48).

PCR was initiated by heating 25 ul Mix 1 (75 pmoles primers, 4 ug adult testis cDNA, 5 umoles dNTPs) and 25 ul Mix 2 [1 unit Fidelity Expand polymerase (Boehringer Mannheim), 5 ul 10X Fidelity Expand Buffer] separately at 96° C. for 20 seconds. Mixes 1 and 2 were then pooled, and the following PCR cycling parameters were used: 96° C., 3 min (1 cycle); 96° C., 30 sec, 55° C.,1 min, 68° C., 2 min (10 cycles); 96° C., 30 sec, 60° C., 1 min, 68° C., 2 min (20 cycles); 72° C., 7 min (1 cycle). After PCR, a single DNA fragment of approximately 0.75 kb was obtained. The DNA fragment was digested with XhoI and XbaI restriction enzymes and cloned into the pCDNA3.1 V5His(B) expression vector (Invitrogen, Carlsbad, Calif.). This vector is named as pCDNA3.1 Ig and contains Fc fragment fused to V5 epitope and 6xHis tag. At the next step a recombinant TEV protease cleavage site was introduced to the N-terminus of the Fc fragment. First, two oligonucleotides were designed, 5′-AATTCTGCAGCGAAAACCTGTATTTTCAGGGT (SEQ ID NO:49) and 5′-TCGAACCCTGAAAATACAGGTTTTCGCTGCAG (SEQ ID NO:50).

These two oligonucleotides were annealed and purified using 20% polyacrylamide gel and ligated into EcoRI and XhoI digested pCDNA3.1Ig, The resulting plasmid was then cut with PstI and PmeI to release a DNA fragment of approximately 0.9 kb, which was ligated into pBlueBac4.5 (Invitrogen, Carlsbad, Calif.) digested with PstI and SmaI. The plasmid construct obtained was named pBIgHis. The Fc fragment was verified by sequence analysis.

### Example 2

Quantitative Expression Analysis of SEQX Nucleic Acids

The quantitative expression of various clones was assessed in 41 normal and 55 tumor samples (see Tables 25 and 26) using TAQMAN® expression analysis. First, 96 RNA samples were normalized to β-actin and GAPDH. RNA (˜50 ng total or ˜1 ng polyA+) was converted to cDNA using the TAQMAN® Reverse Transcription Reagents Kit (PE Biosystems, Foster City, Calif.; cat # N808-0234) and random hexamers according to the manufacturer's protocol. Reactions were performed in 20 ul and incubated for 30 min. at 48° C. cDNA (5 ul) was then transferred to a separate plate for the TAQMAN® reaction using B-actin and GAPDH TAQMAN® Assay Reagents (PE Biosystems; cat. #'s 4310881E and 4310884E, respectively) and TAQMAN® universal PCR Master Mix (PE Biosystems; cat # 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between two samples being represented as 2 to the power of delta CT. The average CT values obtained for β-actin and GAPDH were used to normalize RNA samples. The RNA sample generating the highest CT value required no further diluting, while all other samples were diluted relative to this sample according to their B-actin/GAPDH average CT values.

Normalized RNA (5 ul) was converted to cDNA and analyzed via TAQMAN® using One Step RT-PCR Master Mix Reagents (PE Biosystems; cat. # 4309169) and gene-specific primers according to the manufacturer's instructions. Forward and reverse primer sequences and a probe sequence were designed for each assay. Their and final concentrations were as follows: forward and reverse primers, 900 nM each, and probe, 200 nM. Primers and probes were generated by SYNTHEGEN (Houston, Tex.). Reactions were performed in 25 ul using the following parameters: 30 min. at 48° C.; 10 min. at 95° C.; 15 sec. at 95° C./1 min. at 60° C. (40 cycles). Results were recorded as CT values using a log scale, with the difference in RNA concentration between two samples being represented as 2 to the power of delta CT. Results are plotted as the percent expression relative to the sample exhibiting the highest expression.

TABLE 2
RNA samples from normal tissues/cells used in expression analysis

| Well # | Position | Normal Tissue/Cells | Total/PolyA+ | Source |
|---|---|---|---|---|
| 1 | A1 | Adipose | total | Invitrogen |
| 2 | A2 | adrenal gland | polyA+ | Clontech |
| 3 | A3 | Bladder | total | Invitrogen |
| 4 | A4 | bone marrow | polyA+ | Clontech |
| 5 | A5 | brain (amygdala) | polyA+ | Clontech |
| 6 | A6 | brain (cerebellum) | total | Clontech |
| 7 | A7 | brain (fetal) | polyA+ | Clontech |
| 8 | A8 | brain (hippocampus) | polyA+ | Clontech |
| 9 | A9 | brain | polyA+ | Clontech |
| | | (hypothalamus) | | |
| 10 | A10 | brain | polyA+ | Clontech |
| | | (substantia nigra) | | |
| 11 | A11 | brain (thalamus) | polyA+ | Clontech |
| 12 | A12 | brain (whole) | total | Clontech |
| 13 | B1 | colon (ascending) | total | Yale |
| 14 | B2 | endothelial | polyA+ | dermal |
| | | cells-treated* | | microvascular |
| | | | | cells* |
| 15 | B3 | endothelial cells | total | HUVEC cells |
| 16 | B4 | heart | polyA+ | Clontech |
| 17 | B5 | kidney | polyA+ | Clontech |
| 18 | B6 | kidney (fetal) | polyA+ | Clontech |
| 19 | B7 | liver | polyA+ | Clontech |
| 20 | B8 | liver (fetal) | polyA+ | Clontech |
| 21 | B9 | lung | total | Clontech |
| 22 | B10 | lung (fetal) | polyA+ | Clontech |
| 23 | B11 | lymph node | polyA+ | Clontech |
| 24 | B12 | mammary gland | total | Clontech |
| 25 | C1 | myometrium | total | Yale |
| 26 | C2 | ovary | total | Research |
| | | | | Genetics |
| 27 | C3 | pancreas | polyA+ | Clontech |
| 28 | C4 | pituitary gland | polyA+ | Clontech |
| 29 | C5 | placenta | polyA+ | Clontech |
| 30 | C6 | prostate | polyA+ | Clontech |
| 31 | C7 | salivary gland | polyA+ | Clontech |
| 32 | C8 | skeletal muscle | polyA+ | Clontech |
| 33 | C9 | small intestine | polyA+ | Clontech |
| 34 | C10 | spinal cord | polyA+ | Clontech |
| 35 | C11 | spleen | polyA+ | Clontech |
| 36 | C12 | stomach | total | Clontech |
| 37 | D1 | testis | total | Clontech |
| 38 | D2 | thymus | total | Clontech |
| 39 | D3 | thyroid | polyA+ | Clontech |
| 40 | D4 | trachea | polyA+ | Clontech |
| 41 | D5 | uterus | polyA+ | Clontech |

*dermal microvascular cells treated with factors to induce leukocyte adhesion

TABLE 3
RNA samples from tumor cells used in expression analysis

| Well # | Position | Tissue | Cell Line | Tumor Type | Total/Poly A+ |
|---|---|---|---|---|---|
| 42 | D6 | Breast | MCF-7 | breast carcinoma (pleural effusion) | total |
| 43 | D7 | | MDA-MB-231 | breast carcinoma (pleural effusion) | total |
| 44 | D8 | | BT-549 | breast carcinoma | total |
| 45 | D9 | | T47D | breast carcinoma (pleural effusion) | total |
| 46 | D10 | | MDA-N | breast carcinoma | total |
| 47 | D11 | Ovary | OVCAR-3 | Ovarian carcinoma | total |
| 48 | D12 | | SK-OV-3 | ovarian carcinoma (ascites) | total |
| 49 | E1 | | OVCAR-4 | Ovarian carcinoma | total |
| 50 | E2 | | OVCAR-5 | Ovarian carcinoma | total |
| 51 | E3 | | IGROV-1 | Ovarian carcinoma | total |
| 52 | E4 | | OVCAR-8 | Ovarian carcinoma | total |
| 53 | E5 | Cns | U87-MG | Glioblastoma/astrocytoma | total |
| 54 | E6 | | SW1783 | Astrocytoma (grade III) | total |
| 55 | E7 | | U-118-MG | Gliablastoma/astrocytoma | total |
| 56 | E8 | | SK-N-AS | Neuroblastoma (from BM metastasis) | total |
| 57 | E9 | | SF-539 | Cns | total |
| 58 | E10 | | SNB-75 | Cns | total |
| 59 | E11 | | SNB-19 | Cns | total |
| 60 | E12 | | U251 | Cns | total |
| 61 | F1 | | SF-295 | Cns | total |
| 62 | F2 | GI tract | SW480 | colon carcinoma | total |
| 63 | F3 | | SW620 | lymph node metastasis from SW480 | total |
| 64 | F4 | | HT29 | colon carcinoma (grade I) | total |
| 65 | F5 | | HCT-116 | colon carcinoma | total |
| 66 | F6 | | CaCo-2 | colon carcinoma (grade II) | total |
| 67 | F7 | | NCl-N87 | gastric carcinoma (from liver metastasis) | total |
| 68 | F8 | | HCT-15 | colon carcinoma | total |
| 69 | F9 | | HCC-2998 | colon carcinoma | total |
| 70 | F10 | Kidney | 786-0 | clear cell carcinoma (used clone PRC) | total |
| 71 | F11 | | A498 | renal carcinoma | Total |
| 72 | F12 | | RXF 393 | renal carcinoma | Total |
| 73 | G1 | | ACHN | renal carcinoma | Total |
| 74 | G2 | | UO-31 | renal carcinoma | Total |
| 75 | G3 | | TK-10 | renal carcinoma | Total |
| 76 | G4 | Liver | HepG2 | Hepatoblastoma | Total |
| 77 | G5 | Lung | LX-1 | small cell lung carcinoma | Total |
| 78 | G6 | | NCl-H69 | small cell lung carcinoma | Total |
| 79 | G7 | | SHP-77 | small cell lung ca. (large cell variant) | Total |
| 80 | G8 | | A549 | non-small cell lung carcinoma | Total |
| 81 | G9 | | SW 900 | Squamous cell carcinoma | Total |
| 82 | G10 | | NCl-H596 | Adenosquamous carcinoma | Total |
| 83 | G11 | | NCl-H23 | non-small cell lung ca rcinoma | Total |
| 84 | G12 | | NCl-H460 | large cell lung ca. (pleural effusion) | Total |
| 85 | H1 | | HOP-62 | non-small cell lung carcinoma | Total |
| 86 | H2 | | NCl-H522 | non-small cell lung carcinoma | Total |
| 87 | H3 | Pancreas | CAPAN 2 | Pancreatic carcinoma | Total |
| 88 | H4 | Prostate | PC-3 | prostate ca. (from bone metastasis) | Total |
| 89 | H5 | Skin | Hs688(A).T | Melanoma | Total |
| 90 | H6 | | Hs688(B).T | LN Mestastasis from Hs688(A).T | Total |
| 91 | H7 | | UACC-62 | Melanoma | Total |
| 92 | H8 | | M14 | Melanoma | Total |
| 93 | H9 | | LOX IMVI | Melanoma | Total |
| 94 | H10 | | SK-MEL-5 | melanoma (from axillary node met.) | Total |
| 95 | H11 | | SK-MEL-28 | Melanoma | Total |
| 96 | H12 | | UACC-257 | Melanoma | Total |

### Example 3

Cloning and Expression of 2826468 cDNA in Insect Cells

Based on the predicted reading frame, PCR primers were designed to amplify the coding region for 2826468. The forward primer was 5′-CGC GGA TCC ACC ATG CCC CCA GGA GCT (SEQ ID NO:51), and the reverse primer was 5′-CCG CTC GAG TGA GGT TAA GTT ACC TTT GG (SEQ ID NO:52).

PCR was initiated by heating 25 ul Mix 1 (75 pmoles primers, 4 ug adult bone marrow cDNA, 5 umoles dNTPs) and 25 ul Mix 2 [1 unit Fidelity Expand polymerase (Boehringer Mannheim), 5 ul 10X Fidelity Expand Buffer] separately at 96° C. for 20 seconds. Mixes 1 and 2 were then pooled, and the following PCR cycling parameters were used: 96° C., 3 min (1 cycle); 96° C., 30 sec, 55° C.,1 min, 68° C., 2 min (10 cycles); 96° C., 30 sec, 60° C., 1 min, 68° C., 2 min (20 cycles); 72° C., 7 min (1 cycle). After PCR, a single DNA fragment of approximately 0.8 kb was obtained. The DNA fragment was digested with BamHI and XhoI restriction enzymes, and cloned into the pBIgHis vector (Example 1). The 2826468 insert was verified by DNA sequence analysis. The resulting expression vector for insect cell expression was called pBIgHis2826468.

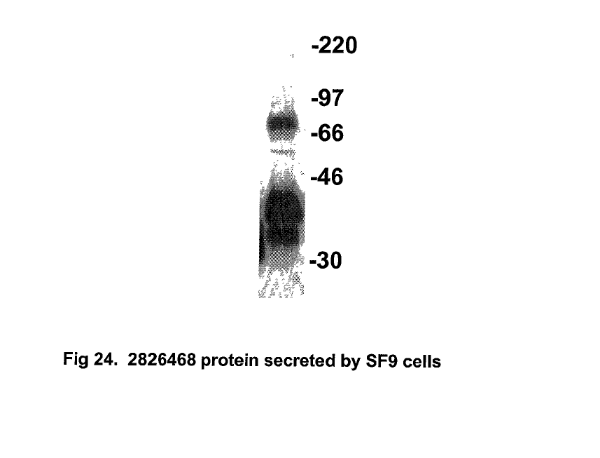

pBIgHis2826468 plasmid DNA was co-transfected with linearized baculovirus DNA (Bac-N-Blue) into SF9 insect cells using liposome-mediated transfer as described by the manufacturer (Invitrogen, Carlsbad, Calif.). Briefly, transfection mixtures containing 4 ug of pBIgHis2826468, 0.5 ug of Bac-N-Blue™ and InsectinPlus™ liposomes were added to 60 mm culture dishes seeded with 2×10⁶ SF9 cells, and incubated with rocking at 27° C. for 4 hours. Fresh culture medium was added and cultures were further incubated for 4 days. The culture medium was then harvested and recombinant viruses were isolated using standard plaque purification procedures. Recombinant viruses expressing β-galactosidase as a marker were readily distinguished from non-recombinant viruses by visually inspecting agarose overlays for blue plaques. Viral stocks were generated by propagation on SF9 cells and screened for expression of 2826468 protein by SDS-PAGE and Western blot analyses (reducing conditions, anti-V5 antibody, Invitrogen, Carlsbad, Calif.) as is shown in FIG. 24. SDS-PAGE analysis reveals that 2826468 is secreted as a 48-kDa protein in SF9 insect cells.

### Example 4

Expression Analysis of 2826468

The quantitative expression of 2826468 in 41 normal and 55 tumor samples was assessed using the methods described in Example 2.

The forward primer was 5′CACGAGGTCAGGAGATCGAGA (SEQ ID NO:53), and the reverse primer was 5′CCCGGCTAATTTTTGTGTGTTTA (SEQ ID NO:54). The probe was 5′ (TET) CTGGCTAACACGGTGAGACCCCATGT (TAMRA) (SEQ ID NO:55).

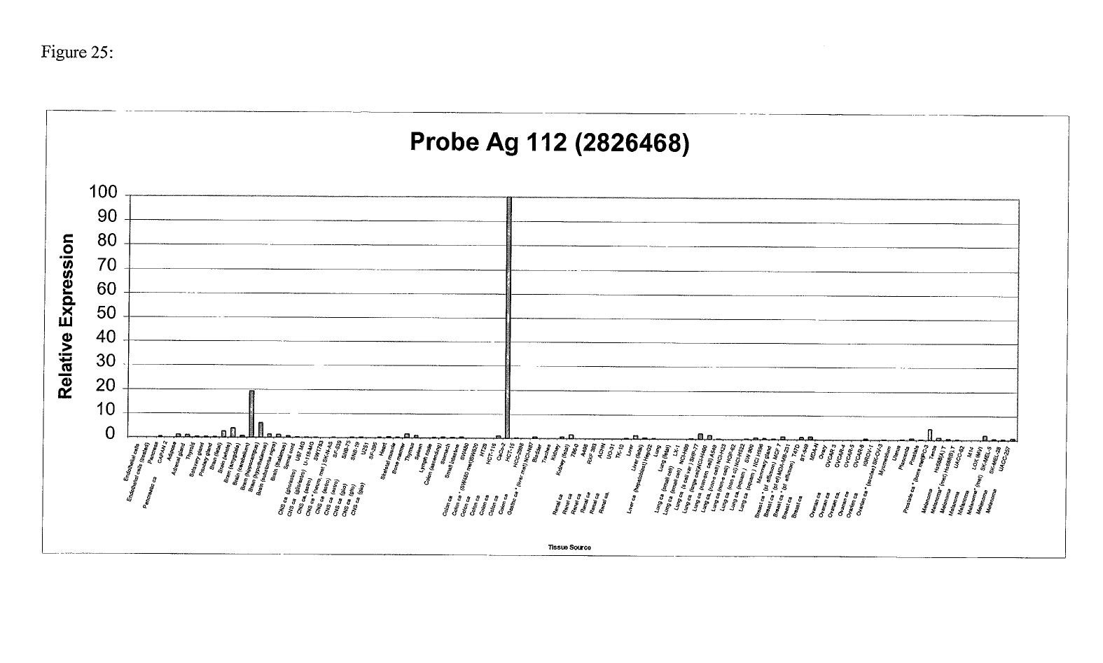

The results are shown in FIG. 25. Expression was detected in normal brain (cerebellum/hippocampus). The gene was also strongly expressed in a colon cancer cell line (CaCo-2) relative to normal colon and all other normal tissues.

### Example 5

Molecular Cloning of 3122461

Both the full length and mature forms of 3122461 were cloned and expressed.

A. Cloning of full length 3122461

Oligonucleotide primers were designed to PCR amplify a DNA segment, representing an ORF, coding for the full length cg3122461. The forward primer included, in addition to the BamHI restriction site, the consensus Kozak sequence. The reverse primer contained an in frame XhoI restriction site. The primers included the following:

2122461 Forward: 5′-GATCCACCATGAGGGGCTCTCAGGAGGTGCTGCTG GCT (SEQ ID NO:56), and 3122461 TOPO Reverse:CTCGAGCTGCAGCTTCTCCTCCAGCAGGTCCAC GCT (SEQ ID NO:57).

PCR reactions were set up using 5 ng human fetal brain cDNA template, 1 microM of each of the 3122461 TOPO Forward and 3122461 TOPO Reverse primers, 5 micromoles dNTP (Clontech Laboratories, Palo Alto Calif.) and 1 microliter of 50xAdvantage-HF 2 polymerase (Clontech Laboratories, Palo Alto Calif.) in 50 microliter volume.

The following reaction conditions were used:

| | |
|---|---|
| a) | 96° C. 3 minutes |
| b) | 96° C. 30 seconds denaturation |
| c) | 70° C. 30 seconds, primer annealing. This temperature was |
| | gradually decreased by 1° C./cycle |
| d) | 72° C. 1 minute extension. |

Repeat steps b-d 10 times

| | |
|---|---|
| e) | 96° C. 30 seconds denaturation |
| f) | 60° C. 30 seconds annealing |
| g) | 72° C. 1 minute extension |

Repeat steps e-g 25 times

| | |
|---|---|
| h) | 72° C. 5 minutes final extension |

A single 820 bp amplified product was detected by agarose gel electrophoresis. The product was isolated by QuiaX (QUIAGEN Inc, Valencia Calif.) in a final volume of 20 microliters.

1) Ten microliters of the isolated fragment was digested with BamHI and XhoI restriction enzymes and ligated into the baculovirus expression vector BacIg (Curagen Corp.). The construct was sequenced and the cloned insert was verified as an ORF coding for the full length 3122461. The construct was called 3122461BacIg.

2) Two microliters of the isolated fragment was directly inserted into the pcDNA3.1-V5His-TOPO vector (Invitrogen, Carlsbad Calif.). The construct was sequenced and the cloned insert was verified as an ORF coding for the full length 3122461, predicted as coding for 273 amino acid residues. The construct was called 3122461pcDNA3.1.

B. Cloning the mature form of cg3122461

By applying the SIGNALP secretory signal prediction method, a signal peptidase cleavage site was identified between residues 22 and 23 for the translated cg3122461 polypeptide. The following oligonucleotide primers were designed to PCR amplify the, SIGNALP predicted, mature form of cg2132461:

2132461Forward:GGA TCC GCC TAC CGG CCC GGC CGT AGG GTG (SEQ ID NO:56), and 2132461Reverse:CTC GAG CGA GTC TTT CTT GCA GGA GCA GGA (SEQ ID NO:57).

PCR reactions were set up using 0.1 ng 3122461pcDNA3.1 plasmid DNA template representing the full length cg3 122461, 1 microM of each of the corresponding primer pairs, 5 micromoles dNTP (Clontech Laboratories, Palo Alto Calif.) and 1 microliter of 50xAdvantage-HF 2 polymerase (Clontech Laboratories, Palo Alto Calif.) in 50 microliter volume. The following reaction conditions were used:

| a) | 96° C. 3 minutes denaturation |
|---|---|
| b) | 96° C. 30 seconds denaturation |
| c) | 60° C. 30 seconds primer annealing |
| d) | 72° C. 1 minute extension |
| repeat steps b-d 15 times | |
| e) | 72° C. 5 minutes final extension |

A single PCR product, with the expected, approximately 750 bp, size, was obtained. The fragment was purified from agarose gel and ligated to pCR2.1 vector (Invitrogen, Carlsbad, Calif.). The cloned insert was sequenced and verified as an open reading frame coding for the predicted mature form of cg3122461between residues 23 and 273 of the full length protein. The clone was called TA-3122461-S315c1

The verified insert was released from TA-3122461-S315c1, by BamHI and XhoI restriction enzyme digestion, and ligated to the pCepSec mammalian expression vector (Curagen Corp.), to the pET28a E.coli expression vector (Novagen, Madison Wis. ) and to the pMelV5His baculovirus expression vector (Curagen Corp.) The recombinants were verified by restriction enzyme digestion.

C. Construction of the pMelV5His expression vector.

The insert was removed from the existing OPG-X pBlueV5His construct (CuraGen Corp.; described in co-owned U.S. Ser. No. 09/422,680, filed Oct. 21, 1999, and incorporated herein by reference) by digesting with NheI and XhoI restriction enzymes linearizing the pBlueV5His vector. pMIgHis (CuraGen Corp.) was digested with NheI and XhoI, releasing a fragment containing the mellitin secretory signal and the consensus Kozak sequence. This fragment was ligated to the linearized pBluV5His vector. The correct structure of the vector was verified by restriction enzyme digestion and PCR analysis.

### Example 6

Expression of 3122461 in Human Embryonic Kidney 293 Cells and in Insect Cells

The expression of 3122461 was examined in human embryonic kidney 293 calls and in insect cells.

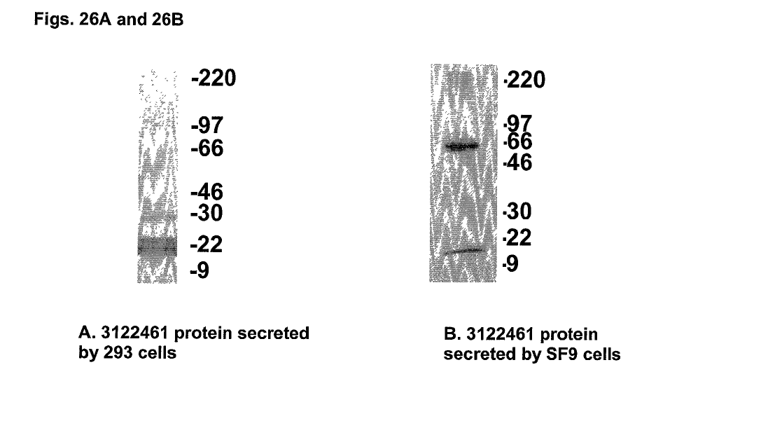

To measure expression in human embryonic kidney 293 cells, the pcDNA3.1V5His3122461 vector was transfected into 293 cells using the LipofectaminePlus reagent following the manufacturer's instructions (Gibco/BRL). The cell pellet and supernatant were harvested 72 hours after transfection and examined for 3122461 expression by Western blotting (reducing conditions) with an anti-V5 antibody. FIG. 26A depicts SDS PAGE analysis in the kidney cells. The 3122461 polypeptide is expressed as a discrete secreted protein around 22-kDa.

Expression in insect cells was examined using a recombinant baculovirus expesssing 3122361. To construct the recombinant baculovirus expressing 3122461, pBIgHis3122461 plasmid DNA was constructed using the pBlgHis vector described in Example 1, and co-transfected with linearized baculovirus DNA (Bac-N-Blue) into SF9 insect cells using liposome-mediated transfer as described by the manufacturer (Invitrogen). Briefly, transfection mixtures containing 4 ug of pBIgHis3122461, 0.5 ug of Bac-N-Blue™ and InsectinPlus™ liposomes were added to 60 mm culture dishes seeded with 2×10⁶ SF9 cells, and incubated with rocking at 27° C. for 4 hours. Fresh culture medium was added and cultures were further incubated for 4 days. The culture medium was then harvested and recombinant viruses were isolated using standard plaque purification procedures. Recombinant viruses expressing β-galactosidase as a marker were readily distinguished from non-recombinant viruses by visually inspecting agarose overlays for blue plaques. Viral stocks were generated by propagation on SF9 cells and screened for expression of 3122461 protein by SDS-PAGE and Western blot analyses (reducing conditions, anti-V5 antibody). The results are shown in FIG. 26B. The figure shows that 3122461 is secreted as a 64-kDa protein.

### Example 7

Subcloning of a GST-3122461 Fusion Protein for Expression in E. coli

A 750 bp DNA fragment of 3122461, which encoded amino acids 23-273 of the predicted 3122461 protein, was amplified by PCR from pcDNA3.1V5His3122461. The forward and reverse primers used contained EcoRI and XhoI sites at the 5′ and 3′ ends, respectively. The PCR product was gel purified, digested with EcoRI and XhoI restriction enzymes and ligated into pGEX 6p-1 (Amersham Pharmacia Biotech, Piscataway, N.J.). This strategy placed the GST sequence of pGEX 6p-1 in-frame at the 5′ end of 3122461. The resulting construct (pGEX6p1-3122461) was introduced into the E. coli strain BL21 for protein production.

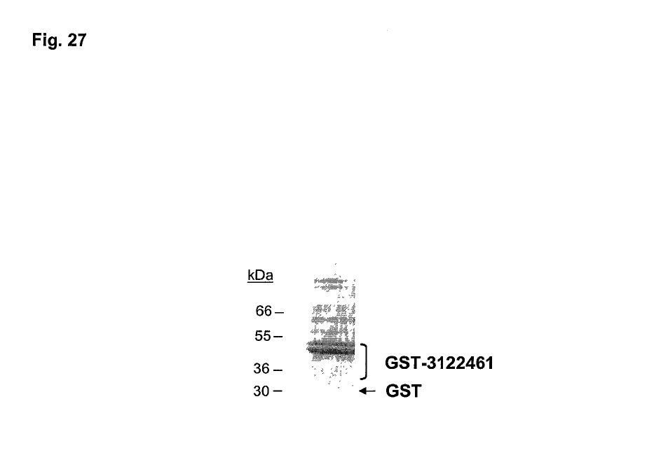

pGEX6p1-3122461 and E. coli BL21 were grown in four liters of LB containing ampicillin (50 mg/ml) at 25C. At an OD₆₀₀ of 0.5 AU, IPTG was added to a final concentration of 1 mM and the culture was further incubated for 4 hours. Cells were then harvested by low-speed centrifugation (5000 rpm in a GS-3 rotor for 15 minutes at 4C.), washed once with 200 ml of phosphate buffered saline (PBS) and resuspended in 200 ml of the same buffer. Cells were disrupted with a microfluidizer (single pass at 10,000 psi) and the insoluble material was removed by low-speed centrifugation (5000 rpm in a GS-3 rotor for 30 minutes at 4C.). The clarified extract was filtered through a 0.2 micron low-protein binding filter and analyzed for protein expression using SDS PAGE and Western analyses (anti-GST antibody, Amersham Pharmacia Biotech). Soluble 3122461 was purified from the clarified extract by batch chromatography using glutathione-sepharose according to the procedures recommended by the manufacturer (Amersham Pharmacia Biotech, Piscataway, N.J.). Specifically, 200 ml of extract was incubated with 1 ml of resin for 1 hour at room temperature with occasional mixing. The unbound proteins were then removed from the resin by washing 2 times with 15 ml of PBS. Bound proteins were eluted from the glutathione beads by incubating the resin with 2.0 ml of reduced glutathione for 30 minutes at room temperature. Eluted 3122461 was dialyzed against 1×10⁶ volumes of PBS (pH 7.4) containing 20% glycerol and at −20° C. A representation of an electrophoretic analysis of the purified GST-3122461 fusion protein is shown in FIG. 27.

### Example 8

Molecular Cloning of 3186754

The extracellular domains of the full-length and secreted forms of 3186754 protein were cloned and expresssed.

A. Cloning the Extracellular Domain of the Full Length 3186754

The cloning procedure was as described in Example 5A, using the following primers:

3186754 N-Forward: 5′-CG GGA TCC ACC ATG GTT GCC CCA AAG CTC CGC TCC T-3′ (SEQ ID NO:58), and

3186754 N-Reverse: 5′-G CTC GAG GCT TAG GCC TGC CTG GGT TCG GAT G -3′ (SEQ ID NO:59).

PCR reactions were set up using 5 ng human testis cDNA template, using the PCR procedure described in Example 5A. A single, 490 bp large, amplified product was detected by agarose gel electrophoresis. The product was isolated and digested with BamHI and XhoI restriction enzymes. The digested product was ligated into the baculovirus expression vector BacIg (Curagen Corp.). The construct was sequenced and the cloned insert was verified as an ORF coding for 3186754 from residues 1 to 163. The construct was named 3186754BacIg.

B. Cloning the Extracellular Domain of the Mature 3186754

The two secretory signal prediction methods, PSORT and SIGNALP, predicted significantly different signal peptidase cleavage sites for cg3186754. While the extracellular domain of the mature cg3186754, predicted by PSORT, is from residue 38 to 163, SIGNALP predicts the signal peptidase cleavage site between residues 61 and 62.

Oligonucleotide primers were designed to PCR amplify both (PSORT and SIGNALP) predicted mature extracellular domain of 3186754.

The following primers were designed to amplify the mature extracellular domain of 3186754 predicted by PSORT:

3186754 Forward PSORT:CTCGTC GGATCC CTC TAT GTG GCC TCG CTT TTG (SEQ ID NO:60), and

3186754 Reverse PSORT:CTCGTC CTC GAG GCT TAG GCC TGC CTG GGT TCG GAT G (SEQ ID NO:61).

The following primers were designed to amplify the mature extracellular domain of 3186754 predicted by SIGNALP:

3186754 Forward SIGP: CTCGTC GGATCC AAG ATG GAC CCC CTA ATC TCT TG (SEQ ID NO:62), and

3186754 Reverse SIGP:CTCGTC CTC GAG GCT TAG GCC TGC CTG GGT TCG GAT G (SEQ ID NO:63).

PCR reactions were run as described in Example 5B. PCR products with the expected sizes, 300 bp for the SIGNALP predicted, and 380 bp for the PSORT predicted segments, were obtained. The fragments were purified from agarose gel and ligated to pCR2.1 vector (Invitrogen, Carlsbad, Calif.). The cloned inserts were sequenced and the inserts were verified as open reading frames coding for the two predicted mature forms. The clones were named 3186754 SIG and 3186754 SORT respectively.

The verified inserts were released from 3186754 SIG and 3186754 SORT by BamHI and XhoI restriction enzyme digestion, and ligated to the pSecV5His mammalian expression vector (Curagen Corp.). The recombinants were verified by restriction enzyme digestion and called 3186754 SIG pSecV5His and 3186754 SORT pSecV5His, respectively.

C. Construction of the mammalian expression vector, pSecV5His

The oligonucleotide primers, PSec-V5-His Forward 5′ CTCGTC CTCGAG GGT AAG CCT ATC CCT AAC (SEQ ID NO:64), and Psec-V5-His Reverse, 5′ CTCGTC GGGCCC CTGATCAGCGGGTTTAAAC (SEQ ID NO:65), were designed to amplify a segment from the pcDNA3.1-V5His (Invitrogen, Carsbad Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis.

### Example 9

Expression of 3186754 in Human Embryonic Kidney 293 Cells and in Insect Cells

Expression of nucleic acid sequences contained in 3186754 was examined in human embryonic kidney and insect cells. A recombinant baculovirus system was used to measure expression in insect cells.

A. Expression in human embryonic kidney 293 cells

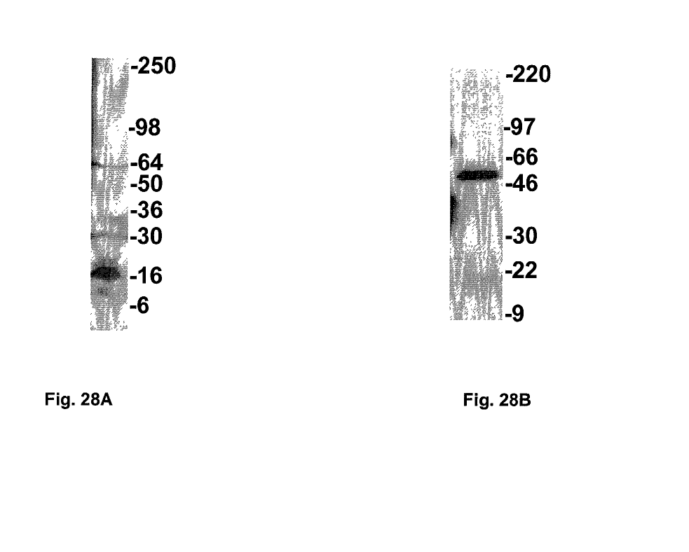

The pSecV5His3186754 vector was transfected into 293 cells using the LipofectaminePlus reagent following the manufacturer's instructions (Gibco/BRL). The cell pellet and supernatant were harvested 72 hours after transfection and examined for 3186754 expression by Western blotting (reducing conditions) with an anti-V5 antibody (Invitrogen, Carlsbad, Calif.). FIG. 28A shows that 3186754 is expressed as an approximately 17-kDa protein in the 293 cells.

B. Construction and isolation of recombinant baculovirus expressing h3186754.

pBIgHis3186754 plasmid DNA was co-transfected with linearized baculovirus DNA (Bac-N-Blue) into SF9 insect cells using liposome-mediated transfer as described by the manufacturer (Invitrogen, Carlsbad, Calif.). Briefly, transfection mixtures containing 4 ug of pBIgHis3186754, 0.5 ug of Bac-N-Blue™ and InsectinPlus™ liposomes were added to 60 mm culture dishes seeded with 2×10⁶ SF9 cells, and incubated with rocking at 27° C. for 4 hours. Fresh culture medium was added and cultures were further incubated for 4 days. The culture medium was then harvested and recombinant viruses were isolated using standard plaque purification procedures. Recombinant viruses expressing β-galactosidase as a marker were readily distinguished from non-recombinant viruses by visually inspecting agarose overlays for blue plaques. Viral stocks were generated by propagation on SF9 cells and screened for expression of 3186754 protein by SDS-PAGE and Western blot analyses (reducing conditions, anti-V5 antibody). FIG. 28B shows that 3186754-Ig is secreted as a 48-kDa protein by SF9 insect cells.

### Example 10

Quantitative Expression Analysis of 3186754

The quantitative expression of 3186754 in 41 normal and 55 tumor samples was assessed as described in Example 2.

The forward primer used was 5′ AGATGATGACGTTGCGAAAGG (SEQ ID NO:66), and the reverse primer was 5′ TACATTGGCCGGAAGATGGA (SEQ ID NO:67).

The probe was 5′ (FAM) CACATCACCCACTCGAAGTCAGCCAC (TAMRA) (SEQ ID NO:68).

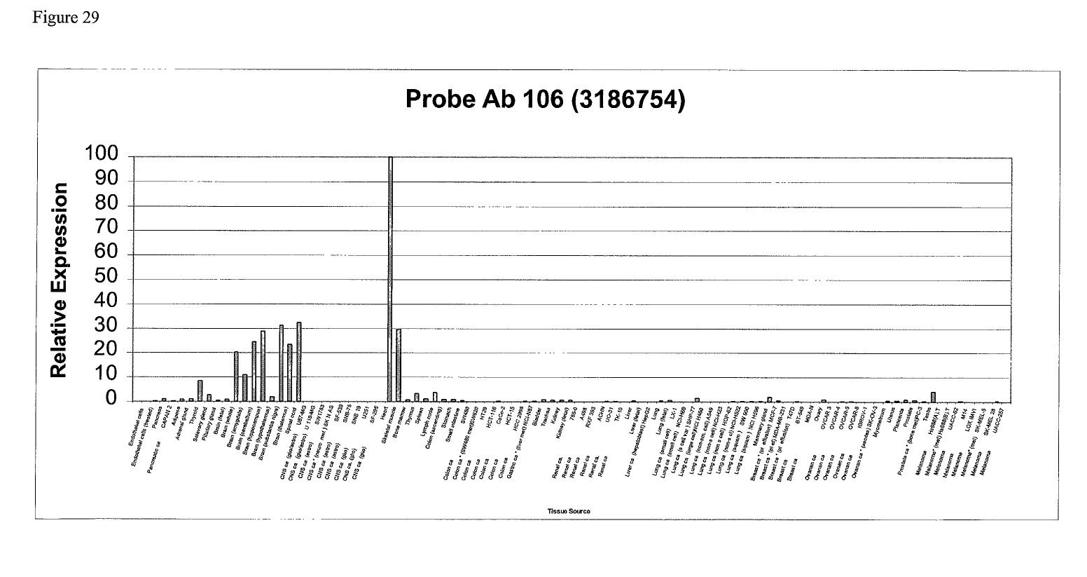

The results are shown in FIG. 29. This gene is most strongly expressed in the heart and is also expressed in skeletal muscle. Expression is also found in normal brain and spinal cord. Only very weak expression was detected in tumor cell lines examined.

### Example 11

Quantitative Expression Analysis of 3277237

The quantitative expression of 3277237 in 41 normal and 55 tumor samples was assessed as described in Example 2.

The forward primer used was 5′ TCCCAAACTTAGTTGCATAGAACCT (SEQ ID NO:69),

the reverse primer was

5′ TCTGTGCCCCGTCCAA (SEQ ID NO:70),

and the probe was

5′ (FAM) TCCTGACCCACGCAGTCCATAAGGA (TAMRA) (SEQ ID NO:71).

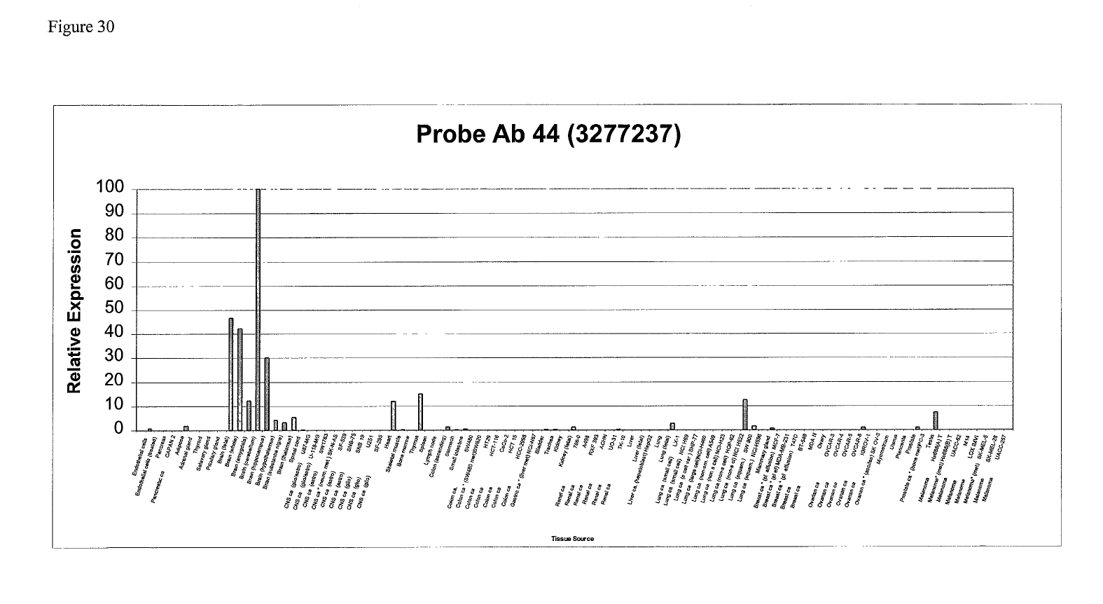

The results of quantitative expression analysis are shown in FIG. 30. The 3277237 gene exhibited strong expression in fetal and adult brain. Especially strong expression is seen in the cerebellum and hippocampus, and moderate expression is seen in the heart and thymus. One lung cancer tumor cell line (HCI-H522) also exhibited a moderate expression of this gene.

### Example 12

Molecular Cloning of 3487483

Both full-length and more forms of the 3487483 were cloned and their expression analyzed.

A. Cloning the full length cg3487483

Oligonucleotide primers were designed to PCR amplify a DNA segment, representing an ORF, coding for the full length 3487483. The forward primer used included, in addition to the BamHI restriction site, the consensus Kozak sequence. The reverse primer contained an in frame XhoI restriction site. The sequences of the primers are the following: 3487483 F-Forward:GCGGATCCACCATGCTGAGCGCCCTGAGCCGGTGCCTCTTCACAC (SEQ ID NO:72), and 3487483 F-Reverse:GCCTCGAGGTGCGAAGCCTCCCCACAGCAGGCTTC (SEQ ID NO:73).

PCR reactions were performed using 5 ng human fetal brain cDNA template, and carried out as described in Example 5A. A single, 300 bp large, amplified product was detected by agarose gel electrophoresis. The product was isolated and digested with BamHI and XhoI restriction enzymes. The digested product was ligated into the baculovirus expression vector BacIg (Curagen Corp.) and into the mammalian expression vector pcDNA3.1V5His (Invitrogen, Carlsbad Calif.). Both constructs were sequenced and the cloned inserts were verified as ORF's coding for cg3487483 from residues 1 to 98. The constructs were called: 3487483BacIg and 3487483pcDNA3.1 respectively.

B. Cloning the mature form of cg3487483

The secretory signal prediction method, PSORT, predicted the signal peptidase cleavage site for cg3487483 between residues 23 and 24.

The following oligonucleotide primers were designed to PCR amplify the, PSORT predicted, mature form of cg3186754. 3487483 SECF: CTCGTC GGATCC TGT ATA AGA CCC ACA GAG GCT C (SEQ ID NO:74), and 3487483 SECR: CTCGTC CTCGAG GTGCGAAGCCTCCCCACAGCAGGCTTC (SEQ ID NO:75).

PCR reactions were set up using 0.1 ng 3487483BacIg plasmid DNA template representing the full length 3487483, and carried out as described in Example 5B. A single PCR product, with the expected, size, 230 bp, was obtained. The fragment was purified from agarose gel and ligated to pCR2.1 vector (Invitrogen, Carlsbad, Calif.). The cloned insert was sequenced and verified as an open reading frame coding for the predicted mature form of 3487483 between residues 24 and 98 of the full length protein. The clone was called TA-3487483-S188A

The verified insert was released from TA-3487483-S188A by BamHI and XhoI restriction enzyme digestion, and ligated to the pSecV5His mammalian expression vector (Curagen Corp.), to the pBADgIII E.coli expression vector (Invitrogen, Carlsbad, Calif.) and to the pMIgHis baculovirus expression vector (Curagen Corp.) The recombinants were verified by restriction enzyme digestion.

### Example 13

Affinity Purification of a 3487483-Fc Chimera

A chimeric 3487483-Ig protein was prepared and characterized.

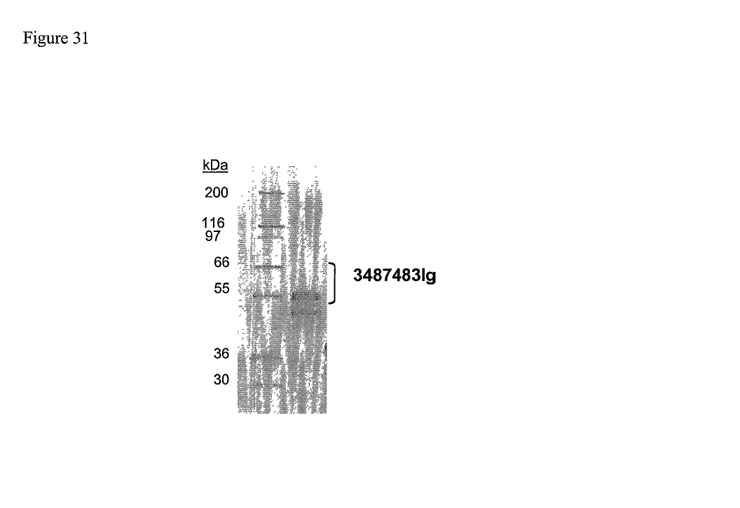

Suspension cultures of SF9 cells were grown in Grace's media containing 5% low IgG fetal calf serum and infected with recombinant 3487483Ig/baculovirus at a multiplicity of infection (MOI) of 0.1. Infected cultures were incubated at 27C. for 4-5 days and the conditioned medium was harvested by low-speed centrifugation (5000 rpm in a GS-3 rotor for 15 minutes at 4C.) to remove cells and debris. The conditioned medium was filtered through a 0.2 micron low-protein binding membrane and analyzed for 3487483Ig production by western analysis using an antibody that detects the V5 epitope (Anti-V5-HRP, Invitrogen). The clarified conditioned medium was then loaded directly onto a 1 ml protein A column (HiTap rProtein A, Amersham Pharmacia) at a flow rate of 1 ml/min at room temperature. Using the Akta Explorer™ FPLC (Amersham Pharmacia), unbound proteins were then washed from the column with 10 ml of 20 mM NaPO₄ (pH 7.0). Bound 3487483Ig was eluted from the column with 25 mM Citrate (pH 2.8) and rapidly neutralized by collecting 0.5 ml fractions in tubes containing 0.5M HEPES buffer (pH 9.1). Fractions containing 3487483Ig were pooled and dialyzed against 1×10⁶ volumes of 20 mM Tris-HCl pH 7.5, 50 mM NaCl. Purified protein samples were stored at −80C. Using this one-step purification protocol, 50 μg were recovered of 3487483Ig protein per liter of conditioned medium with a purity of >85%. SDS PAGE analysis of the fusion protein is shown in FIG. 31.

### Example 14

Construction and Isolation of Recombinant Baculovirus Expressing 3487483

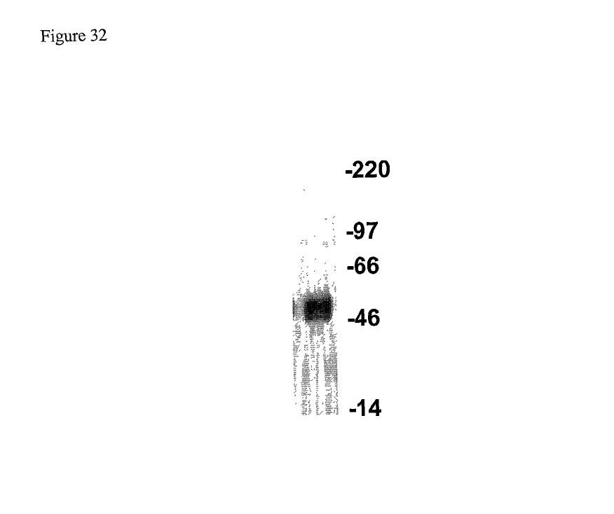

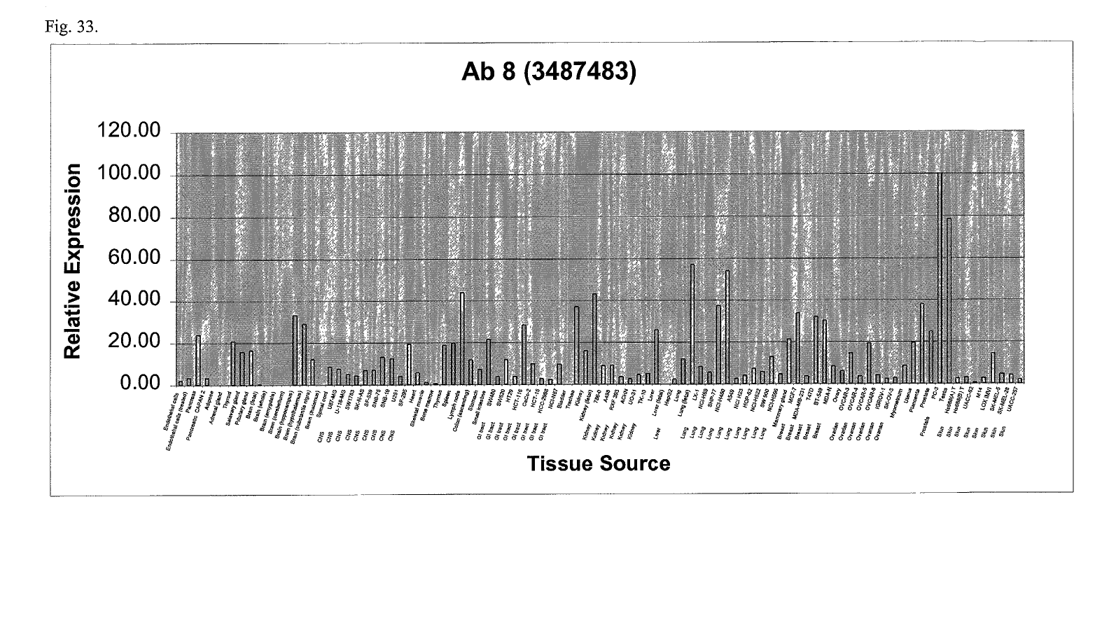

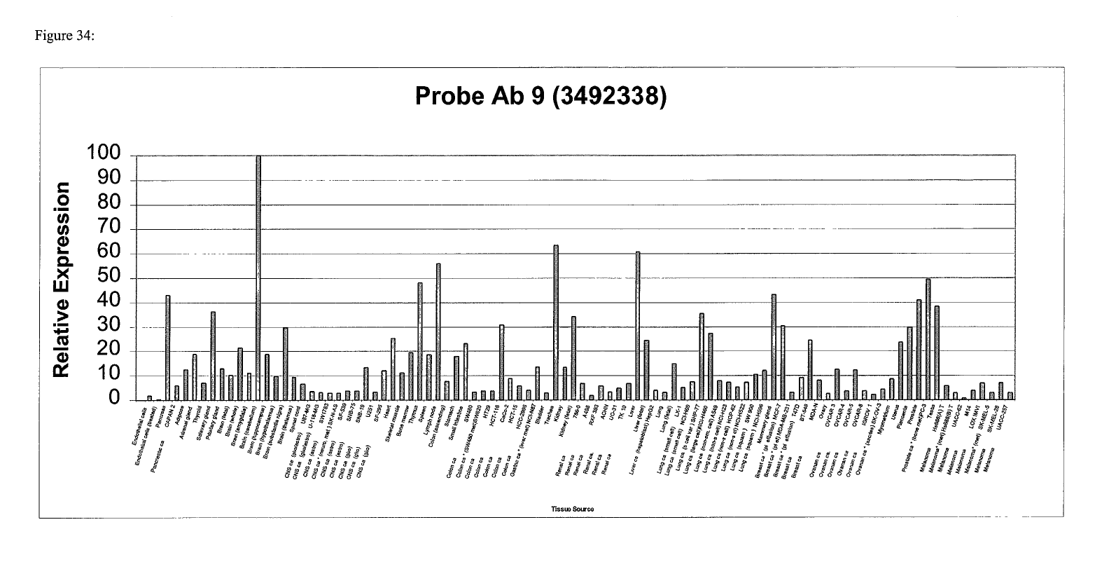

pBIgHis3487483 (renamed from and identical to 3487483Ig/baculovirus in Example 13) was constructed from the pBIgHis baculo expression vector described in Example 1. The resulting plasmid DNA was co-transfected with linearized baculovirus DNA (Bac-N-Blue) into SF9 insect cells using liposome-mediated transfer as described by the manufacturer (Invitrogen, Carlsbad, Calif.). Briefly, transfection mixtures containing 4 ug of pBIgHis3487483, 0.5 ug of Bac-N-Blue™ and InsectinPlus™ liposomes were added to 60 mm culture dishes seeded with 2×10⁶ SF9 cells, and incubated with rocking at 27° C. for 4 hours. Fresh culture medium was added and cultures were further incubated for 4 days. The culture medium was then harvested and recombinant viruses were isolated using standard plaque purification procedures. Recombinant viruses expressing β-galactosidase as a marker were readily distinguished from non-recombinant viruses by visually inspecting agarose overlays for blue plaques. Viral stocks were generated by propagation on SF9 cells and screened for expression of h3487483 protein by SDS-PAGE and Western blot analyses (reducing conditions, anti-V5 antibody, Invitrogen, Carlsbad, Calif.). FIG. 32 shows that 3487483 is secreted as a 48-kDa protein SF9 insect cells.

### Example 15

Quantitative Expression Analysis of 3487483

The quantitative expression of 3487483 in 41 normal and 55 tumor samples was assessed as described in Example 2. Primer and probe sequences included, as the forward primer: 5′ TGTTCTGGGCATGGTGTATAAGA (SEQ ID NO:76); as the reverse primer:

5′ ACAGGGAAAGGGACCCACA (SEQ ID NO:77), and as the probe 5′ (FAM) ACAGCATCACCCGGAGCCTCTGTG (TAMRA) (SEQ ID NO:78).

The results of the expression analysis are shown in FIG. 31. This gene is expressed in many different tissues. Relative to normal tissues, this gene is over-expressed in a couple of lung cancer cell lines (SHP-77 and NCI-H460) and a prostate cancer cell line (PC3).

### Example 16

Quantitative Expression Analysis of 3492338

The quantitative expression of 3492338 in 41 normal and 55 tumor samples was assessed via quantitative expression analysis as described in Example 2. Primer and probe sequences were as follows: forward primer, 5′ GAAAGAAAAGGCATTTAGCAAGGT (SEQ ID NO:79); reverse primer, 5′ GCTTCTCCTCCCCTCTTCTAGG (SEQ ID NO:80); and probe, 5′ (FAM) AAACACAGCGACTCCAGTGCGAGCT (TAMRA) (SEQ ID NO:81).

The results of the expression analysis are shown in FIG. 11. This gene is expressed in many tissues. Highest expression was observed in the brain (cerebellum). This gene is over-expressed in one colon cancer cell line (HCT-116) relative to normal colon tissue and in two lung cancer cell lines (SHP-77 and NCI-H460) relative to normal lung tissue.

### Example 17

Expression Analysis of 3540920

The quantitative expression of 3540920 in 41 normal and 55 tumor samples was assessed as described in Example 2.

Primer and probe sequences were as follows: forward primer, 5′ CAGCATCCGCCCAAAAGTT (SEQ ID NO:82); reverse primer, 5′ GGCCTGTCCATCATGTATTGTCT (SEQ ID NO:83), and probe 5′ (FAM) CTCCCAT GACAGCAGGAAGCAGCC (TAMRA) (SEQ ID NO:84).

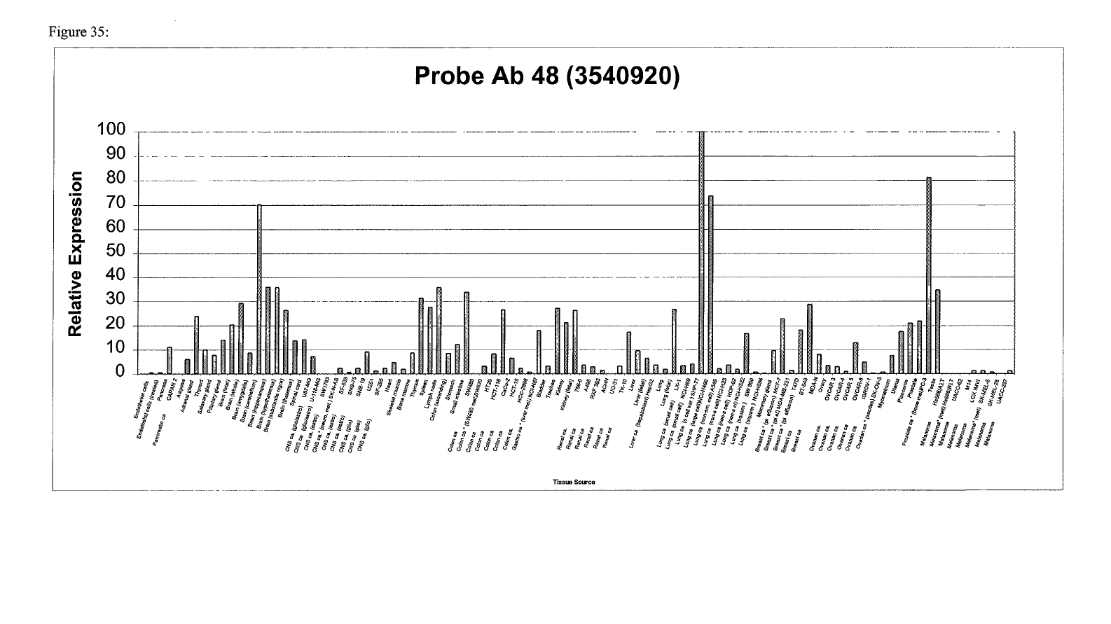

The results are shown in FIG. 35. Expression was detected in many different tissues, with strongest expression seen in the brain (cerebellum). Over-expression was observed in three lung cancer cell lines (SHP-77, NCI-H460 and NCI-H522) relative to normal lung tissue and in one prostate cell line (PC3) relative to normal prostate tissue.

### Example 18

Expression Analysis of 3903091

The quantitative expression of 3903091 in 41 normal and 55 tumor samples was assessed as described in Example 2.

Primer and probe sequences were as follows: forward primer, 5′ AAGTGAGTTCAAAACCGCTAGGA (SEQ ID NO:85); reverse primer, 5′TGTGGCAGGCAACACCAA (SEQ ID NO:86), and probe 5′ (FAM) TCCGCTTACGCTATGCGATGACCA (TAMRA) (SEQ ID NO:87).

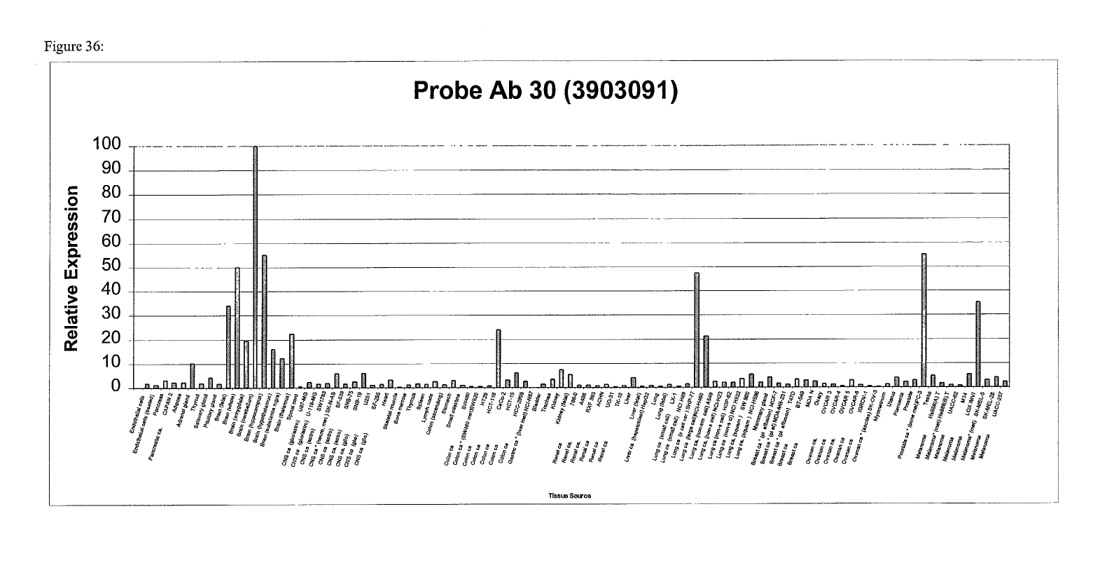

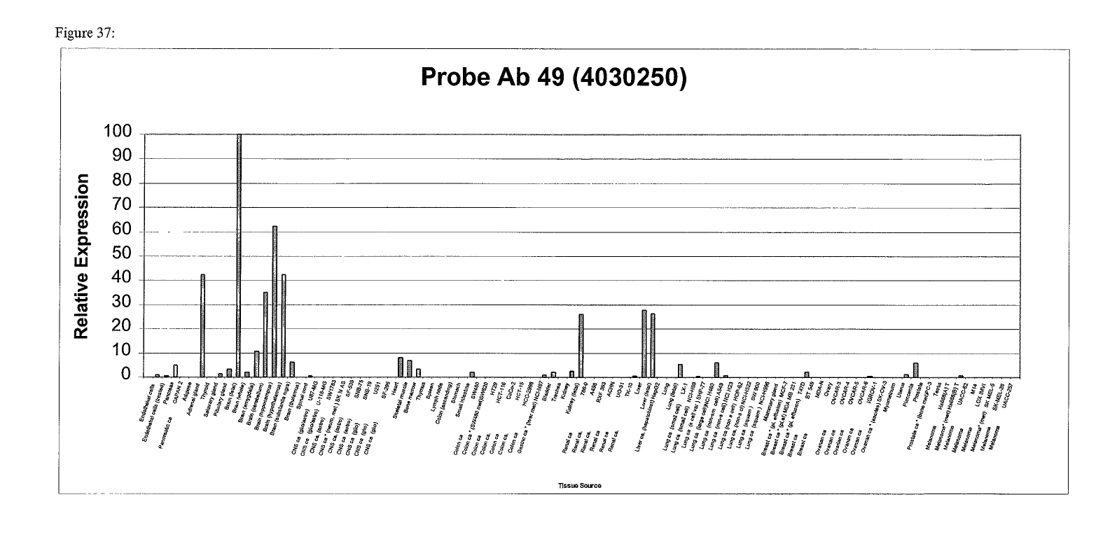

The results of expression analyses are shown in FIG. 36. The gene exhibited brain-specific expression, being expressed in a variety of locations in the brain (amygdala, cerebellum, hippocampus, hypothalamus, thalamus and substantia nigra). Overexpression was seen in one colon cancer cell line (HCT-116) relative to normal colon tissue, in two lung cancer cell lines (SHP-77 and NCI-H460) relative to normal lung tissue, in one prostate cancer line (PC3) relative to normal prostate tissue and in one melanoma (LOX IMVI).

### Example 19

Expression Analysis of 4030250

The quantitative expression of 4030250 in 41 normal and 55 tumor samples was assessed as described in Example 2.

Primer and probe sequences were as follows: forward primer, 5′ CACCAGGAGATAGGCAATGCA (SEQ ID NO:88); reverse primer, 5′ TGGCGGCCACCATCA (SEQ ID NO:89), and, probe: 5′ (TET) CCCCCGGCAGCAAGAAATCCA (TAMRA) (SEQ ID NO:90).

The results are shown in FIG. 27. Expression was observed in all fetal tissues examined (brain, liver and kidney) as well as in adult tissues. The adult tissues included the liver, adrenal gland and regions of the brain (cerebellum, hippocampus and hypothalamus). Very weak expression of this gene is seen in tumor cell lines.

Equivalents

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. An isolated nucleic acid comprising a sequence encoding a SECX polypeptide at least 90% identical to a polypeptide comprising an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 150, 152, and 154.

2. The nucleic acid of claim 1, wherein said nucleic acid encodes a polypeptide comprising an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 150, 152, and 154.

3. The nucleic acid of claim 1, wherein said nucleic acid encodes a polypeptide consisting of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 150, 152, and 154.

4. The nucleic acid of claim 1, wherein said nucleic acid comprises a nucleic acid sequence selected from the group consisting of SEQ ID NOs: 147, 92, 148, 95, 96, 97, 100, 103, 106, 111, 112, 115, 116, 119, 122, 125, 128, 131, 134, 137, 140, 143, and 144.

5. The nucleic acid of claim 1, wherein said nucleic acid comprises a nucleic acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 149, 151, 153, 155, 156, and 157.

6. The nucleic acid of claim 1, wherein said nucleic acid is DNA.

7. The nucleic acid of claim 1, wherein said nucleic acid is RNA.

8. An isolated nucleic acid comprising a nucleic acid selected from the group consisting of SEQ ID NOs: 147, 92, 148, 95, 96, 97, 100, 103, 106, 111, 112, 115, 116, 119, 122, 125, 128, 131, 134, 137, 140, 143, and 144, or its complement.

9. The nucleic acid of claim 8, wherein said nucleic acid comprises a nucleic acid selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 149, 151, 153, 155, 156, and 157, or its complement.

10. An isolated nucleic acid comprising a nucleotide sequence complementary to at least a portion of a nucleic acid selected from the group consisting of SEQ ID NOs: 147, 92, 148, 95, 96, 97, 100, 103, 106, 111, 112, 115, 116, 119, 122, 125, 128, 131, 134, 137, 140, 143, and 144.

11. The nucleic acid of claim 10, wherein said nucleic acid comprises a nucleic acid complementary to a portion of a nucleic acid selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 149, 151, 153, 155, 156, and 157.

12. A vector comprising the nucleic acid of claim 1.

13. A cell comprising the vector of claim 12

14. The cell of claim 13, wherein said cell is a prokaryotic cell.

15. The cell of claim 13, wherein said cell is a eukaryotic cell.

16. A pharmaceutical composition comprising the nucleic acid of claim 1 and a pharmaceutically acceptable carrier.

17. A substantially purified polypeptide comprising an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 150, 152, and 154.

18. The polypeptide of claim 17, wherein said polypeptide consists of an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 150, 152, and 154.

19. A pharmaceutical composition comprising the polypeptide of claim 17 and a pharmaceutically acceptable carrier.

20. An antibody which binds specifically to the polypeptide of claim 17.

21. The antibody of claim 20, wherein said antibody is a polyclonal antibody.

22. The antibody of claim 21, wherein said antibody is a monoclonal antibody.

23. A kit comprising the antibody of claim 20 and, optionally, a negative control antibody.

24. A pharmaceutical composition comprising the antibody of claim 20 and a pharmaceutically acceptable carrier.

25. A method of producing a SECX polypeptide, the method comprising:
(a) providing the cell of claim 13;
(b) culturing said cell under conditions sufficient to express said SECX polypeptide; and
(c) recovering said SECX polypeptide,
thereby producing said SECX polypeptide.

26. The method of claim 25, wherein said cell is a prokaryotic cell.

27. The method of claim 25, wherein said cell is a eukaryotic cell.

28. A method of diagnosing a pathological condition associated with aberrant SECX expression or activity in a subject, the method comprising:
(a) providing a protein sample from said subject;
(b) measuring the amount of SECX polypeptide in said subject sample; and
(c) comparing the amount of SECX polypeptide in said subject protein sample to the amount of SECX polypeptide in a control protein sample,
wherein an alteration in the amount of SECX polypeptide in said subject protein sample relative to the amount of SECX polypeptide in said control protein sample indicates the subject has said pathological condition.

29. The method of claim 28, wherein said SECX polypeptide is detected using the antibody of claim 20.

30. The method of claim 28, wherein said pathological condition is cancer.

31. A method of diagnosing a pathological condition associated with aberrant SECX expression or activity in a subject, the method comprising:
(a) providing a nucleic acid sample from said subject;
(b) measuring the amount of SECX nucleic acid in said subject nucleic acid sample; and
(c) comparing the amount of SECX nucleic acid sample in said subject nucleic acid to the amount of SECX nucleic acid in a control sample,
wherein an alteration in the amount of SECX nucleic acid in said sample relative to the amount of SECX in said control sample indicates the subject has said pathological condition.

32. The method of claim 31, wherein the measured SECX nucleic acid is SECX RNA.

33. The method of claim 31, wherein the measured SECX nucleic acid is SECX DNA.

34. The method of claim 31, wherein the pathological condition is cancer.

35. The method of claim 31, wherein the SECX nucleic acid is measured by using one or more nucleic acids which amplify the nucleic acid of claim 1.

36. A method of diagnosing a pathological condition associated with aberrant SECX expression or activity in a subject, the method comprising:
(a) providing a nucleic acid sample from said subject;
(b) identifying at least a portion of the nucleotide sequence of a SECX nucleic acid in said subject nucleic acid sample; and
(c) comparing the SECX nucleotide sequence of said subject sample to a SECX nucleotide sequence of a control sample,
wherein an alteration in the SECX nucleotide sequence in said sample relative to the SECX nucleotide sequence in said control sample indicates the subject has said pathological condition.

37. A method of treating or preventing or delaying a pathological condition associated with aberrant SECX expression or activity in a subject, the method comprising administering to a subject in which such treatment or prevention or delay is desired the nucleic acid of claim 1 in an amount sufficient to treat, prevent, or delay said pathological condition in said subject.

38. The method of claim 39, wherein said pathological condition is cancer.

39. A method of treating or preventing or delaying a pathological condition associated with aberrant SECX expression or activity in a subject, the method comprising administering to a subject in which such treatment or prevention or delay is desired the polypeptide of claim 20 in an amount sufficient to treat, prevent, or delay said pathological condition in said subject.

40. A method of treating or preventing or delaying a pathological condition associated with aberrant SECX expression or activity in a subject, the method comprising administering to a subject in which such treatment or prevention or delay is desired the antibody of claim 24 in an amount sufficient to treat, prevent or delay a pathological condition in said subject.

41. A method for identifying a compound that binds SECX protein, the method comprising:
a) contacting SECX protein with a compound; and
b) determining whether said compound binds SECX protein.

42. The method of claim 41, wherein binding of said compound to SECX protein is determined by a protein binding assay.

43. A compound identified by the method of claim 41.

44. A method for identifying a compound that binds a nucleic acid encoding a SECX protein, the method comprising:
a) contacting said nucleic acid encoding SECX protein with a compound; and
b) determining whether said compound binds said nucleic acid molecule.

45. A compound identified by the method of claim 44.

46. A method for identifying a compound that modulates the activity of a SECX protein, the method comprising:
a) contacting SECX protein with a compound; of
b) determining whether SECX protein activity has been modulated.

47. A compound identified by the method of claim 46.
